SEQUENCE LISTING (38 sequences)

SEQ ID NO: 1 - protein, 195 aa, Homo sapiens
TQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTR

SEQ ID NO: 2 - protein, 124 aa, Homo sapiens
TVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARV

SEQ ID NO: 3 - protein, 169 aa, Homo sapiens
CYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLA

SEQ ID NO: 4 - protein, 142 aa, Homo sapiens
CHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLT

SEQ ID NO: 5 - protein, 214 aa, Artificial sequence
  Sequence is synthesized
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 449 aa, Artificial sequence
  sequence is synthesized
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 7 - protein, 214 aa, Artificial sequence
  Sequence is synthesized
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 448 aa, Artificial sequence
  Sequence is synthesized
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 9 - DNA, 37 bp, Homo sapiens
gccgaagcttgccaccatgggcagcgacccgagcgcg

SEQ ID NO: 10 - DNA, 41 bp, Homo sapiens
gctgtctagagttcaccgtccgtgccagtgccacctcctcc

SEQ ID NO: 11 - DNA, 37 bp, Homo sapiens; identical to SEQ ID NO: 9

SEQ ID NO: 12 - DNA, 31 bp, Homo sapiens
gctgtctagagacccactcctgcagcgagcg

SEQ ID NO: 13 - DNA, 33 bp, Homo sapiens
cgagtcgacctcgaggccagccctctgacgtcc

SEQ ID NO: 14 - DNA, 30 bp, Homo sapiens
ggcacgcgtcgtcagagggctggctctctg

SEQ ID NO: 15 - DNA, 27 bp, Homo sapiens
ggcctcgagggcctggcctgccaccag

SEQ ID NO: 16 - DNA, 30 bp, Homo sapiens; identical to SEQ ID NO: 14

SEQ ID NO: 17 - protein, 8 aa, Artificial sequence
  Sequence is synthesized
DYKDDDDK

SEQ ID NO: 18 - DNA, 21 bp, Homo sapiens
ccaccatctgacctttagctt

SEQ ID NO: 19 - DNA, 66 bp, Homo sapiens
gatccgccaccatctgacctttagcttcaagagagctaaaggtcagatggtggttttttgctagcg

SEQ ID NO: 20 - DNA, 66 bp, Homo sapiens
aattcgctagcaaaaaaccaccatctgacctttagctctcttgaagctaaaggtcagatggtggcg

SEQ ID NO: 21 - protein, 32 aa, Homo sapiens
PINCTHSCVDLDDKGCPAEQRASPASPLTSIV

SEQ ID NO: 22 - protein, 5 aa, Homo sapiens
DLDDK

SEQ ID NO: 23 - protein, 8 aa, Homo sapiens
PAEQRASP

SEQ ID NO: 24 - protein, 42 aa, Homo sapiens
EEGACQPINCTHSCVDLDDKGCPAEQRASPLTSIVSAVVGIL

SEQ ID NO: 25 - protein, 26 aa, Homo sapiens
EEGACQPINCTHSPLTSIVSAVVGIL

SEQ ID NO: 26 - protein, 37 aa, Homo sapiens
EEGACQPINCTHSCVGCPAEQRASPLTSIVSAVVGIL

SEQ ID NO: 27 - protein, 33 aa, Homo sapiens
EEGACQPINCTHSCVDLDDKGCPAEQRAVVGIL

SEQ ID NO: 28 - protein, 16 aa, Homo sapiens
LTSIVSTRDKTHTCPP

SEQ ID NO: 29 - protein, 16 aa, Homo sapiens
LTSIVSTRDKTHTCPP

SEQ ID NO: 30 - protein, 16 aa, Homo sapiens
LTSIVSTRDKTHTCPP

SEQ ID NO: 31 - protein, 16 aa, Homo sapiens
LTSIVSTRDKTHTCPP

SEQ ID NO: 32 - protein, 8 aa, Homo sapiens
KYALADAS

SEQ ID NO: 33 - protein, 41 aa, Homo sapiens
CPINCTHSCVDLDDKGCPAEQRASPLTSIVSAVVGILLVVV

SEQ ID NO: 34 - protein, 27 aa, Homo sapiens
DLDDKGCPAEQRASPLTSIVSAVVGIL

SEQ ID NO: 35 - protein, 9 aa, Homo sapiens
PTNGPKIPS

SEQ ID NO: 36 - protein, 14 aa, Homo sapiens
CLGQTLVLIGKTLT

SEQ ID NO: 37 - protein, 18 aa, Homo sapiens
CIYYPWTGHSTLPQHART

SEQ ID NO: 38 - protein, 8 aa, Homo sapiens
CIGLMDRT

(19) United States
(12) Patent Application Publication
(10) Pub. No.: US 2006/0177448 A1
(43) Pub. Date: Aug. 10, 2006

(54) Inhibiting HER2 shedding with matrix metalloprotease antagonists

(75) Inventors: Kendall D. Carey, South San Francisco, CA (US); Ralph Schwall, Pacifica, CA (US); Mark Sliwkowski, San Carlos, CA (US); Gail Colbern Schwall, Pacifica, CA (US)

Correspondence Address: GENENTECH, INC., 1 DNA WAY, SOUTH SAN FRANCISCO, CA, 94080, US

(73) Assignee: Genentech, Inc., South San Francisco, CA (US)

(21) Appl. No.: 11/351,811

(22) Filed: Feb. 9, 2006

Related U.S. Application Data
(60) Provisional application No. 60/651,348, filed on Feb. 9, 2005.

(51) Int. Cl. G01N 33/574; A61K 39/395

(52) U.S. Cl. 424/146.1; 435/7.23

(57) ABSTRACT

The present application describes using antagonists of matrix metalloproteases (MMPs), especially of MMP-15, for inhibiting HER2 shedding.

This is a non-provisional application claiming priority under 35 USC § 119 to provisional application no. 60/651,348 filed Feb. 9, 2005, the entire disclosure of which is hereby incorporated by reference.

### FIELD OF THE INVENTION

The present invention concerns using antagonists of matrix metalloproteases (MMPs), especially of MMP-15, for inhibiting HER2 shedding.

### BACKGROUND OF THE INVENTION

The HER family of receptor tyrosine kinases are important mediators of cell growth, differentiation and survival. The receptor family includes four distinct members including epidermal growth factor receptor (EGFR, ErbB1, or HER1), HER2 (ErbB2 or p185^{neu}), HER3 (ErbB3) and HER4 (ErbB4 or tyro2).

EGFR, encoded by the erbB1 gene, has been causally implicated in human malignancy. In particular, increased expression of EGFR has been observed in breast, bladder, lung, head, neck and stomach cancer as well as glioblastomas. Increased EGFR receptor expression is often associated with increased production of the EGFR ligand, transforming growth factor alpha (TGF-α), by the same tumor cells resulting in receptor activation by an autocrine stimulatory pathway. Baselga and Mendelsohn Pharmac. Ther. 64:127-154 (1994). Monoclonal antibodies directed against the EGFR or its ligands, TGF-α and EGF, have been evaluated as therapeutic agents in the treatment of such malignancies. See, e.g., Baselga and Mendelsohn, supra; Masui et al. Cancer Research 44:1002-1007 (1984); and Wu et al J. Clin. Invest. 95:1897-1905 (1995).

The second member of the HER family, p185^{neu}, was originally identified as the product of the transforming gene from neuroblastomas of chemically treated rats. The activated form of the neu proto-oncogene results from a point mutation (valine to glutamic acid) in the transmembrane region of the encoded protein. Amplification of the human homolog of neu is observed in breast and ovarian cancers and correlates with a poor prognosis (Slamon et al., Science, 235:177-182 (1987); Slamon et al., Science, 244:707-712 (1989); and U.S. Pat. No. 4,968,603). To date, no point mutation analogous to that in the neu proto-oncogene has been reported for human tumors. Overexpression of HER2 (frequently but not uniformly due to gene amplification) has also been observed in other carcinomas including carcinomas of the stomach, endometrium, salivary gland, lung, kidney, colon, thyroid, pancreas and bladder. See, among others, King et al., Science, 229:974 (1985); Yokota et al., Lancet: 1:765-767 (1986); Fukushige et al., Mol Cell Biol., 6:955-958 (1986); Guerin et al., Oncogene Res., 3:21-31 (1988); Cohen et al., Oncogene, 4:81-88 (1989); Yonemura et al., Cancer Res., 51:1034 (1991); Borst et al., Gynecol. Oncol., 38:364 (1990); Weiner et al., Cancer Res., 50:421425 (1990); Kern et al., Cancer Res., 50:5184 (1990); Park et al., Cancer Res., 49:6605 (1989); Zhau et al., Mol. Carcinog., 3:254-257 (1990); Aasland et al. Br. J. Cancer 57:358-363 (1988); Williams et al. Pathobiology 59:46-52 (1991); and McCann et al., Cancer, 65:88-92 (1990). HER2 may be overexpressed in prostate cancer (Gu et al. Cancer Lett. 99:185-9 (1996); Ross et al. Hum. Pathol. 28:827-33 (1997); Ross et al. Cancer 79:2162-70(1997); and Sadasivan et al. J. Urol. 150:126-31 (1993)).

Antibodies directed against the rat p185^{neu} and human HER2 protein products have been described.

Drebin and colleagues have raised antibodies against the rat neu gene product, p185^{neu} See, for example, Drebin et al, Cell 41:695-706 (1985); Myers et al, Meth. Enzym. 198:277-290 (1991); and WO94/22478. Drebin et al. Oncogene 2:273-277 (1988) report that mixtures of antibodies reactive with two distinct regions of p185^{neu} result in synergistic anti-tumor effects on neu-transformed NIH-3T3 cells implanted into nude mice. See also U.S. Pat. No. 5,824,311 issued Oct. 20, 1998.

Hudziak et al, Mol. Cell. Biol. 9(3):1165-1172 (1989) describe the generation of a panel of HER2 antibodies which were characterized using the human breast tumor cell line SK-BR-3. Relative cell proliferation of the SK-BR-3 cells following exposure to the antibodies was determined by crystal violet staining of the monolayers after 72 hours. Using this assay, maximum inhibition was obtained with the antibody called 4D5 which inhibited cellular proliferation by 56%. Other antibodies in the panel reduced cellular proliferation to a lesser extent in this assay. The antibody 4D5 was further found to sensitize HER2-overexpressing breast tumor cell lines to the cytotoxic effects of TNF-α. See also U.S. Pat. No. 5,677,171 issued Oct. 14, 1997. The HER2 antibodies discussed in Hudziak et al are further characterized in Fendly et al Cancer Research 50:1550-1558 (1990); Kotts et al. In Vitro 26(3):59A (1990); Sarup et al Growth Regulation 1:72-82 (1991); Shepard et al J. Clin. Immunol. 11(3):117-127 (1991); Kumar et al Mol. Cell. Biol. 11(2):979-986 (1991); Lewis et al. Cancer Immunol. Immunother. 37:255-263 (1993); Pietras et al. Oncogene 9:1829-1838 (1994); Vitetta et al. Cancer Research 54:5301-5309 (1994); Sliwkowski et al. J. Biol. Chem. 269(20):14661-14665 (1994); Scott et al J. Biol. Chem. 266:14300-5 (1991); D'souza et al Proc. Natl. Acad Sci. 91:7202-7206 (1994); Lewis et al. Cancer Research 56:1457-1465 (1996); and Schaefer et al. Oncogene 15:1385-1394 (1997).

A recombinant humanized version of the murine HER2 antibody 4D5 (huMAb4D5-8, rhuMAb HER2, trastuzumab or HERCEPTIN®; U.S. Pat. No. 5,821,337) is clinically active in patients with HER2-overexpressing metastatic breast cancers that have received extensive prior anti-cancer therapy (Baselga et al., J. Clin. Oncol. 14:737-744 (1996)). Trastuzumab received marketing approval from the Food and Drug Administration September 25, 1998 for the treatment of patients with metastatic breast cancer whose tumors overexpress the HER2 protein.

Other HER2 antibodies with various properties have been described in Tagliabue et al Int. J. Cancer 47:933-937 (1991); McKenzie et al Oncogene 4:543-548 (1989); Maier et al. Cancer Res. 51:5361-5369 (1991); Bacus et al. Molecular Carcinogenesis 3:350-362 (1990); Stancovski et al PNAS (USA) 88:8691-8695 (1991); Bacus et al. Cancer Research 52:2580-2589 (1992); Xu et al Int. J. Cancer 53:401408 (1993); WO94/00136; Kasprzyk et al Cancer Research 52:2771-2776 (1992);Hancock et al Cancer Res. 51:4575-4580 (1991); Shawver et al Cancer Res. 54:1367-1373 (1994); Arteaga et al Cancer Res. 54:3758-3765 (1994); Harwerth et al J. Biol. Chem. 267:15160-15167 (1992); U.S. Pat. No. 5,783,186; and Klapper et al. Oncogene 14:2099-2109 (1997).

Homology screening has resulted in the identification of two other HER receptor family members; HER3 (U.S. Pat. Nos. 5,183,884 and 5,480,968 as well as Kraus et al PNAS (USA) 86:9193-9197 (1989)) and HER4 (EP Pat Appln No 599,274; Plowman et al., Proc. Natl. Acad. Sci. USA, 90:1746-1750 (1993); and Plowman et al., Nature, 366:473-475 (1993)). Both of these receptors display increased expression on at least some breast cancer cell lines.

The HER receptors are generally found in various combinations in cells and heterodimerization is thought to increase the diversity of cellular responses to a variety of HER ligands (Earp et al. Breast Cancer Research and Treatment 35: 115-132 (1995)). EGFR is bound by six different ligands; epidermal growth factor (EGF), transforming growth factor alpha (TGF-α), amphiregulin, heparin binding epidermal growth factor (HB-EGF), betacellulin and epiregulin (Groenen et al. Growth Factors 11:235-257 (1994)). A family of heregulin proteins resulting from alternative splicing of a single gene are ligands for HER3 and HER4. The heregulin family includes alpha, beta and gamma heregulins (Holmes et al., Science, 256:1205-1210 (1992); U.S. Pat. No. 5,641,869; and Schaefer et at. Oncogene 15:1385-1394 (1997)); neu differentiation factors (NDFs), glial growth factors (GGFs); acetylcholine receptor inducing activity (ARIA); and sensory and motor neuron derived factor (SMDF). For a review, see Groenen et al. Growth Factors 11:235-257 (1994); Lemke, G. Molec. & Cell. Neurosci. 7:247-262 (1996) and Lee et al. Pharm. Rev. 47:51-85 (1995). Recently three additional HER ligands were identified; neuregulin-2 (NRG-2) which is reported to bind either HER3 or HER4 (Chang et al. Nature 387 509-512 (1997); and Carraway et al Nature 387:512-516 (1997)); neuregulin-3 which binds HER4 (Zhang et al PNAS (USA) 94(18):9562-7 (1997)); and neuregulin4 which binds HER4 (Harari et al. Oncogene 18:2681-89 (1999)) HB-EGF, betacellulin and epiregulin also bind to HER4.

While EGF and TGFα do not bind HER2, EGF stimulates EGFR and HER2 to form a heterodimer, which activates EGFR and results in transphosphorylation of HER2 in the heterodimer. Dimerization and/or transphosphorylation appears to activate the HER2 tyrosine kinase. See Earp et al., supra. Likewise, when HER3 is co-expressed with HER2, an active signaling complex is formed and antibodies directed against HER2 are capable of disrupting this complex (Sliwkowski et al., J. Biol. Chem., 269(20):14661-14665 (1994)). Additionally, the affinity of HER3 for heregulin (HRG) is increased to a higher affinity state when co-expressed with HER2. See also, Levi et al., Journal of Neuroscience 15: 1329-1340 (1995); Morrissey et al., Proc. Natl. Acad Sci. USA 92: 1431-1435 (1995); and Lewis et al., Cancer Res., 56:1457-1465 (1996) with respect to the HER2-HER3 protein complex. HER4, like HER3, forms an active signaling complex with HER2 (Carraway and Cantley, Cell 78:5-8 (1994)).

The HER signaling network is depicted in FIG. 4.

Patent publications related to HER antibodies include: U.S. Pat. No. 5,677,171, U.S. Pat. No. 5,720,937, U.S. Pat. No. 5,720,954, U.S. Pat. No. 5,725,856, U.S. Pat. No. 5,770,195, U.S. Pat. No. 5,772,997, U.S. Pat. No. 6,165,464, U.S. Pat. No. 6,387,371, U.S. Pat. No. 6,399,063, US2002/0192211A1, U.S. Pat. No. 6,015,567, U.S. Pat. No. 6,333,169, U.S. Pat. No. 4,968,603, U.S. Pat. No. 5,821,337, U.S. Pat. No. 6,054,297, U.S. Pat. No. 6,407,213, U.S. Pat. No. 6,719,971, U.S. Pat. No. 6,800,738, US2004/0236078A1, U.S. Pat. No. 5,648,237, U.S. Pat. No. 6,267,958, U.S. Pat. No. 6,685,940, U.S. Pat. No. 6,821,515, WO98/17797, U.S. Pat. No. 6,127,526, U.S. Pat. No. 6,333,398, U.S. Pat. No. 6,797,814, U.S. Pat. No. 6,339,142, U.S. Pat. No. 6,417,335, U.S. Pat. No. 6,489,447, WO99/31140, US2003/0147884A1, US2003/0170234A1, US2005/0002928A1, U.S. Pat. No. 6,573,043, US2003/0152987A1, WO99/48527, US2002/0141993A1, WO01/00245, US2003/0086924, US2004/0013667AI, WO00/69460, WO01/00238, WO0/15730, U.S. Pat. No. 6,627,196B1, U.S. Pat. No. 6,632,979B1, WO01/00244, US2002/0090662A1, WO01/89566, US2002/0064785, US2003/0134344, WO 04/24866, US2004/0082047, US2003/0175845A1, WO03/087131, US2003/0228663, WO2004/008099A2, US2004/0106161, WO02004/048525, US2004/0258685A1, U.S. Pat. No. 5,985,553, U.S. Pat. No. 5,747,261, U.S. Pat. No. 4,935,341, U.S. Pat. No. 5,401,638, U.S. Pat. No. 5,604,107, WO 87/07646, WO 89/10412, WO 91/05264, EP 412,116 B1, EP 494,135 B1, U.S. Pat. No. 5,824,311, EP 444,181 B1, EP 1,006,194 A2, US 2002/0155527A1, WO 91/02062, U.S. Pat. No. 5,571,894, U.S. Pat. No. 5,939,531, EP 502,812 B1, WO 93/03741, EP 554,441 B1, EP 656,367 A1, U.S. Pat. No. 5,288,477, U.S. Pat. No. 5,514,554, U.S. Pat. No. 5,587,458, WO 93/12220, WO 93/16185, U.S. Pat. No. 5,877,305, WO 93/21319, WO 93/21232, U.S. Pat. No. 5,856,089, WO 94/22478, U.S. Pat. No. 5,910,486, U.S. Pat. No. 6,028,059, WO 96/07321, U.S. Pat. No. 5,804,396, U.S. Pat. No. 5,846,749, EP 711,565, WO 96/16673, U.S. Pat. No. 5,783,404, U.S. Pat. No. 5,977,322, U.S. Pat. No. 6,512,097, WO 97/00271, U.S. Pat. No. 6,270,765, U.S. Pat. No. 6,395,272, U.S. Pat. No. 5,837,243, WO 96/40789, U.S. Pat. No. 5,783,186, U.S. Pat. No. 6,458,356, WO 97/20858, WO 97/38731, U.S. Pat. No. 6,214,388, U.S. Pat. No. 5,925,519, WO 98/02463, U.S. Pat. No. 5,922,845, WO 98/18489, WO 98/33914, U.S. Pat. No. 5,994,071, WO 98/45479, U.S. Pat. No. 6,358,682 B1, US 2003/0059790, WO 99/55367, WO 01/20033, US 2002/0076695 A1, WO 00/78347, WO 01/09187, WO 01/21192, WO 01/32155, WO 01/53354, WO 01/56604, WO 01/76630, WO02/05791, WO 02/11677, U.S. Pat. No. 6,582,919, US2002/0192652A1, US 2003/0211530A1, WO 02/44413, US 2002/0142328, U.S. Pat. No. 6,602,670 B2, WO 02/45653, WO 02/055106, US 2003/0152572, US 2003/0165840, WO 02/087619, WO 03/006509, WO03/012072, WO 03/028638, US 2003/0068318, WO 03/041736, EP 1,357,132, US 2003/0202973, US 2004/0138160, U.S. Pat. No. 5,705,157, U.S. Pat. No. 6,123,939, EP 616,812 B1, US 2003/0103973, US 2003/0108545, U.S. Pat. No. 6,403,630 B1, WO 00/61145, WO 00/61185, U.S. Pat. No. 6,333,348 B1, WO 01/05425, WO 01/64246, US 2003/0022918, US 2002/0051785 A1, U.S. Pat. No. 6,767,541, WO 01/76586, US 2003/0144252, WO 01/87336, US 2002/0031515 A1, WO 01/87334, WO 02/05791, WO 02/09754, US 2003/0157097, US 2002/0076408, WO 02/055106, WO 02/070008, WO 02/089842 and WO 03/86467.

The HER2 extracellular domain (ECD) is proteolytically shed from breast carcinoma cells in culture (Petch et al., Mol. Cell. Biol. 10:2973-2982 (1990); Scott et al, Mol. Cell. Biol. 13:2247-2257 (1993); and Lee and Maihle, Oncogene 16:3243-3252(1998)), and is found in the serum of some cancer patients (Leitzel et al., J. Clin. Oncol. 10:1436-1443 (1992)). HER2 ECD may be a serum marker of metastatic breast cancer (Leitzel et al., J. Clin. Oncol. 10:1436-1443 (1992)), and may allow escape of HER2 overexpressing tumors from immunological control (Baselga et al, J. Clin. Oncol. 14:737-744 (1997), Brodowicz et al., Int. J. Cancer 73:875-879 (1997)). Shed HER2 ECD serum levels represent an independent marker of poor clinical outcome in patients with HER2 overexpressing metastatic breast cancer (Ali et al, Clin. Chem. 48:1314-1320 (2002); Molina et al., Clin. Cancer Res. 8:347-353 (2002)).

A truncated extracellular domain of HER2 is also the product of a 2.3 kb alternative transcript generated by use of a polyadenylation signal within an intron (Scott et al, Mol. Cell. Biol. 13:2247-2257 (1993)). The alternative transcript was first identified in the gastric carcinoma cell line, MKN7 (Yamamoto et al, Nature 319:230-234 (1986); and Scott et al., Mol. Cell. Biol. 13:2247-2257 (1993)) and the truncated receptor was located within the perinuclear cytoplasm rather than secreted from these tumor cells (Scott et al, Mol. Cell. Biol. 13:2247-2257 (1993)).

Another alternatively spliced product of HER2, called “herstatin,” has also been identified (Doherty et al, Proc. Natl. Acad. Sci. 96:10869-10874 (1999); Azios et al, Oncogene 20:5199-5209 (2001); Justman and Clinton, J. Biol. Chem. 277:20618-20624 (2002)). This protein consists of subdomains I and II from the extracellular domain followed by a unique C-terminal sequence encoded by intron 8.

Another mechanism that may account for poor clinical outcome in HER2 overexpressing tumors is suggested by the observation that, in some HER2 overexpressing tumor cells, the receptor is processed by an unknown met alloprotease (or met alloproteinase) to yield a truncated, membrane-associated receptor (sometimes referred to as a “stub” and also known as p95), and a soluble extracellular domain (also known as ECD, ECD105, or p105).

As with other HER receptors, loss of the extracellular ligand binding domain renders the HER2 intracellular membrane-associated domain a constitutively active tyrosine kinase. It has therefore been postulated that the processing of the HER2 ECD creates a constitutively active receptor that can directly deliver growth and survival signals to the cancer cell. See, U.S. Pat. No. 6,541,214 (Clinton), and US Patent Appln No. 2004/0247602A1 (Friedman et al.)

Saez et al. Clin Cancer Res. 12(2): 424-431 (January, 2006) report that patients whose tumors express high levels of p95 have a significantly worse outcome than patients who do not. At present p95 level can only be determined by Western blot.

### SUMMARY OF THE INVENTION

In a first aspect, the invention concerns a method for inhibiting HER2 shedding comprising treating a HER2 expressing cell with a matrix met alloprotease (MMP) antagonist in an amount effective to inhibit HER2 shedding.

In addition, the invention provides a method for reducing HER2 extracellular domain (ECD) serum level in a mammal, comprising administering a matrix met alloprotease (MMP) antagonist to the mammal in an amount effective to reduce the HER2 ECD serum level in the mammal.

In yet another aspect, a method for treating cancer in a mammal is provided, which comprises administering a matrix met alloprotease (MMP) antagonist to the mammal in an amount effective to treat the cancer.

Also, the invention concerns a method for treating a HER inhibitor-resistant cancer in a mammal comprising administering to the mammal a matrix met alloprotease (MMP) antagonist in an amount effective to treat the cancer.

In yet a further aspect, the invention relates to a method for reducing p95 HER2 level in a cell comprising exposing the cell to a matrix met alloprotease (MMP) antagonist in an amount effective to reduce the p95 HER2 level.

The invention also concerns a method of diagnosis (or prognosis) comprising evaluating MMP-15 (MT2-MMP) in a sample from a cancer patient, wherein elevated MMP-15 level or activity indicates the patient has an elevated p95 HER2 and/or shed HER2 serum level, and/or will have a poor clinical outcome. Preferably, MMP-15 level (protein or nucleic acid) is evaluated in the method and is used to identify patients with a poor prognosis, or who will have a poor clinical outcome. Optionally, the patient's cancer further displays HER expression, amplification, or activation, most preferably HER2 overexpression or amplification.

### BRIEF DESCRIPTION OF THE DRAWINGS

FIG. 1 provides a schematic of the full length HER2 protein structure, and amino acid sequences for Domains I-IV (SEQ ID NOs. 1-4, respectively) of the extracellular domain thereof.

FIGS. 2A and 2B show the amino acid sequences of trastuzumab light chain (FIG. 2A; SEQ ID No. 5) and heavy chain (FIG. 2B; SEQ ID No. 6), respectively.

FIGS. 3A and 3B show the amino acid sequences of pertuzumab light chain (FIG. 3A; SEQ ID NO. 7) and heavy chain (FIG. 3B; SEQ ID NO. 8). CDRs are shown in bold. Calculated molecular mass of the light chain and heavy chain are 23,526.22 Da and 49,216.56 Da (cysteines in reduced form). The carbohydrate moiety is attached to Asn 299 of the heavy chain.

FIG. 4 depicts the HER signaling network.

FIG. 5 illustrates trastuzumab inhibition of HER2 ECD shedding from HER2 overexpressing breast cancer cell lines (SKBR3, MT474) compared to nonoverexpressing breast cancer cell line (MCF-7).

FIG. 6 illustrates differences in p95 HER2 and shed HER2 ECD levels in MMTVHER2 transgenic tumors (f2:1282 tumors, trastuzumab-sensitive; and Of5 tumors, trastuzumab-resistant).

FIG. 7 depicts the strategy used to identify HER2 sheddase.

FIG. 8 illustrates experiments which demonstrated sheddase had properties of a met alloprotease.

FIG. 9 shows expression of MMPs in MMTV-HER2 tumors and cell lines. MMP-15 is a candidate for explaining differences between f2:1282 tumors which are trastuzumab-sensitive, and Of5 tumors which are trastuzumab-resistant.

FIG. 10 reflects interaction between flag-HER2 and MMP-15.

FIG. 11 shows interaction between flagHER2-f2: 1282 and flagHER2-Fo5 and MMP-15. Differences between f2:1282 and Fo5 are not explainable by differential binding of mutants to MMP-15.

FIG. 12 illustrates somatic mutations found in MMTVHER2 transgenic mice. The sequences are: sheddase site (SEQ ID NO. 23), wild-type (WT) (SEQ ID NO. 24), splice (SEQ ID NO. 25), Fo5 (SEQ ID NO. 26), and f2:3078.10 (SEQ ID NO. 27).

FIG. 13 depicts results of the in vitro sheddase assay. gDHER29(DIV)-IgG is a substrate for the catalytic domains of MMP-15, MMP-16, MMP-19, and MMP-25. The sequences for protease digests are MMP-15 (SEQ ID NO. 28), MMP-16 (SEQ ID NO. 29), MMP-19 (SEQ ID NO. 30), MMP-25 (SEQ ID NO. 31), all other (SEQ ID NO.32); and for the HER2 ECD C-terminal site (SEQ ID NO. 33).

FIG. 14 illustrates results of experiment demonstrating MMP-15 does not clip other HER receptors. The sequences demonstrating sequence variation near transmembrane domain are HER2 (SEQ ID NO. 34), EGFR (SEQ ID NO. 35), HER3 (SEQ ID NO. 36), HER4 (Jma) (SEQ ID NO. 37), and HER4 (Jmb) (SEQ ID NO. 38).

FIG. 15 shows MMP-15 “full length” clips HER2(+)-IgG.

FIG. 16 demonstrates an experiment which indicated p95HER2 is constitutively phosphorylated, but must heterdimerize with HER3 to activate Akt.

FIG. 17 shows MMP-15 RNA inhibitor (RNAi) reduces HER2 ECD shedding and p95 HER2 levels in SKBR3 and BT474 cells.

FIG. 18 illustrates how trastuzumab-mediated growth inhibition in SKBR3 cells is independent of inhibiting HER2 shedding.

FIG. 19 demonstrates that inhibition of met alloprotease activity in Fo5 xenograft tumors reduces HER2 shedding and inhibits p95HER2 levels.

FIG. 20 depicts members of the matrix met alloproteinase (MMP) family. MMP family members are grouped according to domain structure. The abbreviations used in this figure are: PRE, pre-domain; PRO, pro-domain; CAT, catalytic domain; H, hinge; HEM, hemopexin domain; F, furin-cleavage consensus domain; FN, fibronectin-like domain; GPI, glycophosphatidyl inositol anchor; TM, transmembrane domain; Ig, immunoglobulin-like domain; CA, cysteine array; CL, collagen-like domain.

### DETAILED DESCRIPTION OF THE PREFERRED EMBODIMENTS

I. Definitions

The terms “matrix met alloprotease” or “MMP” herein refer to a protein which is a member of a matrix met alloprotease (MMP) superfamily, dependent on Zn or Ca for activity. MMP herein includes the preproprotein, mature protein and variant forms thereof. See, also, FIG. 20 herein for examples of MMPs with various domains. MMPs are reviewed in Wagenaar-Miller et al. Cancer and Metastasis Reviews 23: 119-135 (2004).

A “membrane-tethered MMP” or “MT-MMP” herein is a MMP as defmed above, where the MMP is capable of being attached to a cell membrane via either a transmembrane (TM) domain or a glycophosphatidyl inositol (GPI) anchor. Examples of MT-MMPs anchored by a transmembrane domain herein include MT1-MMP (MMP-14), MT2-MMP (MMP-15), MT3-MMP (MMP-16), MT5-MMP (MMP-24). Examples of MT-MMPs anchored by a GPI anchor include MT4-MMP (MMP-17), and MT6-MMP (MMP-25). MMP-15 is the preferred MT-MMP herein.

“MT2-MMP” and “MMP-15” are synonyms herein and describe the preproprotein NP_{—}002415 in the NCB1 database, the mature protein comprising amino acids 132-699 thereof, as well as variant forms thereof. Known substrates for MMP-15 include collagen, fibronectin, CD44, and complement. MMP-15 is upregulated in some cancers, and overexpression of this protease enhances tumor invasion and tumor cell growth.

A “MMP antagonist” is an agent that binds to and/or interferes to some extent with proteolytic activity of at least one MMP. Preferably, the MMP antagonist selectively binds to, or inhibits, the MMP, without significantly binding to, or inhibiting, other proteases, such as proteases in the ADAM (a disintegrin and met alloprotease) family. Examples of MMP antagonists herein include antibodies that bind to a MMP, small molecule inhibitors, pseudopeptides that mimic MMP substrates, nonpeptidic molecules that bind the catalytic zinc of MMPs, isolated natural tissue inhibitors of MMPs (TIMPs), nucleic acid inhibitors, such as RNA; or antisense inhibitors, etc.

A “MT-MMP antagonist” is an agent that binds to and/or interferes to some extent with proteolytic activity of at least one MT-MMP. Preferably, the MT-MMP antagonist selectively binds to, or inhibits, the MT-MMP, without significantly binding to, or inhibiting, other proteases (including other MMPs that are not membrane-tethered). Examples of MT-MMP antagonists include antibodies that bind to a MT-MMP, small molecule inhibitors, pseudopeptides that mimic MT-MMP substrates, nonpeptidic molecules that bind the catalytic zinc of a MT-MMP, isolated natural tissue inhibitors of MT-MMPs, MT-MMP nucleic acid inhibitors, such as RNA; or antisense inhibitors, etc.

A “MMP-15 antagonist” is an agent that binds to and/or interferes to some extent with proteolytic activity of MMP-15. Preferably, the MMP-15 antagonist selectively binds to, or inhibits, MMP-15 without significantly binding to, or inhibiting, other proteases (including MMPs other than MMP-15). Examples of MMP-15 antagonists include antibodies that bind to MMP-15, small molecule inhibitors, pseudopeptides that mimic MMP-15 substrates, nonpeptidic molecules that bind the catalytic zinc of MMP-15, isolated natural tissue inhibitors of MMP-15, MMP-15 nucleic acid inhibitors such as RNA; or antisense inhibitors etc.

By “elevated MMP level” is meant an amount of MMP in a biological sample, such as a tumor sample, that exceeds the normal amount of the MMP, for instance the amount in a normal, or non-tumor, sample of the same tissue type. Such “normal amount” of MMP (e.g. of MMP-15) includes no or undetectable amount of MMP-15. Elevated MMP levels can be determined in various ways, including those which measure MMP protein or MMP nucleic acid.

A “HER receptor” is a receptor protein tyrosine kinase which belongs to the HER receptor family and includes EGFR, HER2, HER3 and HER4 receptors. The HER receptor includes native sequence HER receptor, and variants thereof. Preferably the HER receptor is native sequence human HER receptor.

A “full length” HER receptor comprises an extracellular domain, which may bind an HER ligand and/or dimerize with another HER receptor molecule; a lipophilic transmembrane domain; an intracellular tyrosine kinase domain; and a carboxyl-terminal signaling domain harboring several tyrosine residues which can be phosphorylated.

The terms “ErbB1,” “HER1”, “epidermal growth factor receptor” and “EGFR” are used interchangeably herein and refer to EGFR as disclosed, for example, in Carpenter et al. Ann. Rev. Biochem. 56:881-914 (1987), including variant forms thereof (e.g. a deletion mutant EGFR as in Humphrey et al. PNAS (USA) 87:42074211 (1990)).

The expressions “ErbB2” and “HER2” are used interchangeably herein and refer to human HER2 protein described, for example, in Semba et al., PNAS (USA) 82:6497-6501 (1985) and Yamamoto et al. Nature 319:230-234 (1986) (Genebank accession number X03363), as well as variant forms thereof, such as alternatively spliced forms (Siegel et al. EMBO J. 18(8):2149-2164 (1999)).

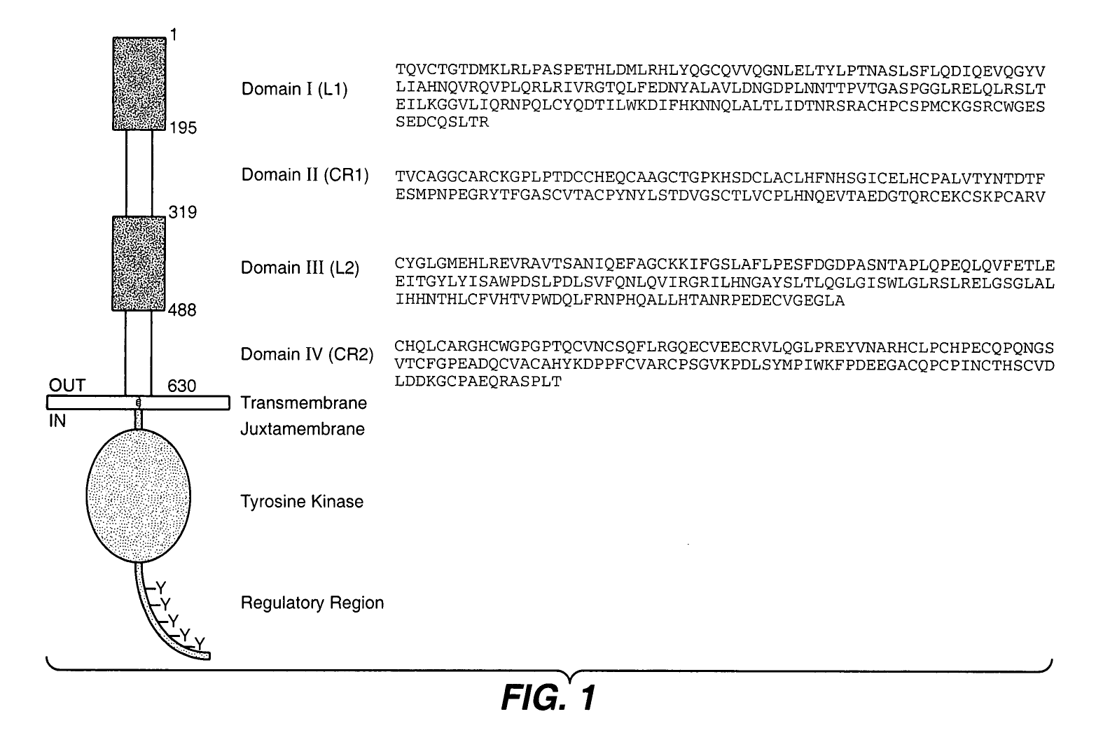

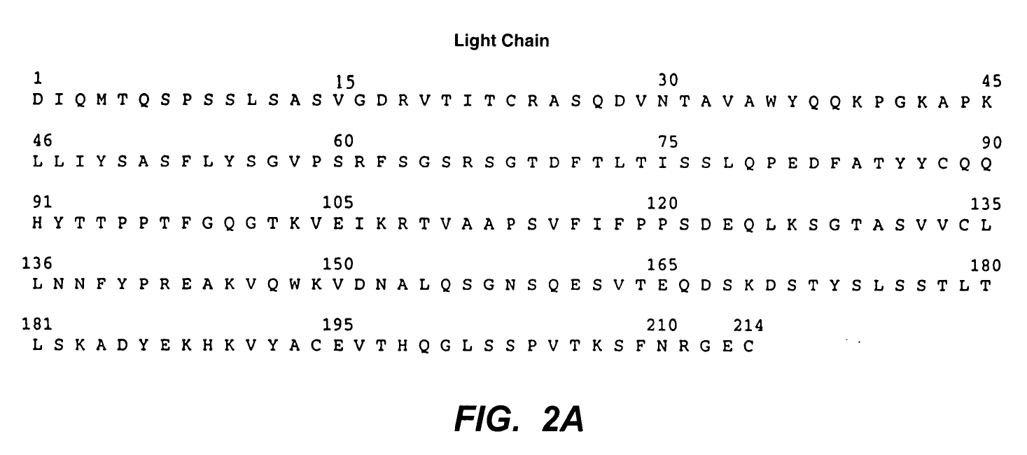

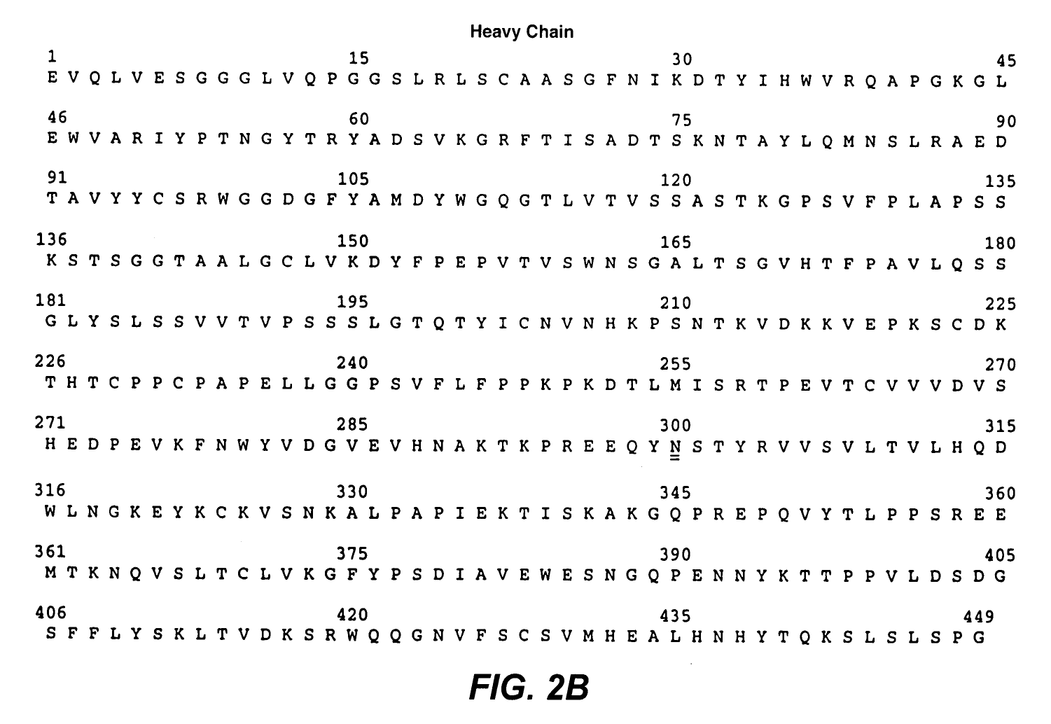

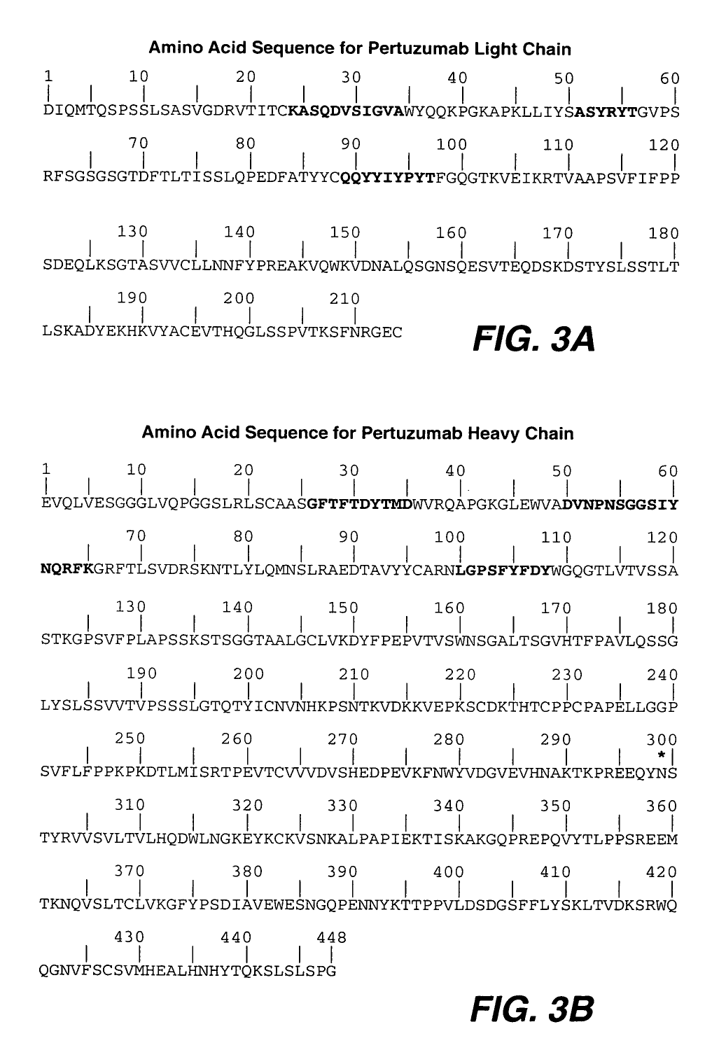

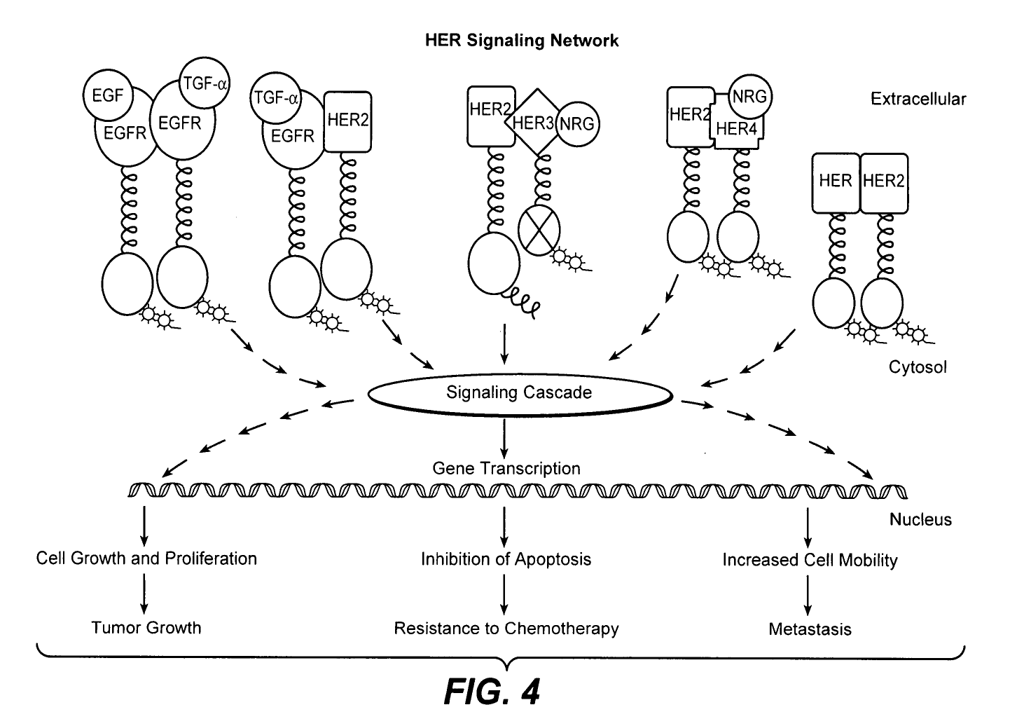

Herein, “HER2 extracellular domain” or “HER2 ECD” refers to a domain of HER2 that is outside of a cell, either anchored to a cell membrane, or in circulation, including fragments thereof. In one embodiment, the extracellular domain of HER2 may comprise four domains: “Domain I” (amino acid residues from about 1-195; SEQ ID NO: 1), “Domain II” (amino acid residues from about 196-319; SEQ ID NO:2), “Domain III” (amino acid residues from about 320488: SEQ ID NO:3), and “Domain IV” (amino acid residues from about 489-630; SEQ ID NO:4) (residue numbering without signal peptide). See Garrett et al. Mol. Cell. 11: 495-505 (2003), Cho et al. Nature 421: 756-760 (2003), Franklin et al. Cancer Cell 5:317-328 (2004), and Plowman et al. Proc. Natl. Acad Sci. 90:1746-1750 (1993), as well as FIG. 1 herein.

Herein, “HER2 shedding” refers to release of a soluble extracellular domain (ECD) fragment of HER2 from the cell surface of a cell which expresses HER2. Such shedding may be caused by proteolytic cleavage of cell surface HER2 resulting in release of an ECD fragment from the cell surface, or the soluble ECD or fragment thereof may be encoded by an alternate transcript.

By “shed HER2 serum level” is meant the amount of HER2 ECD present in the serum or circulation of a mammal. Such levels can be evaluated by various techniques including those described in: Ali et al. Clin. Chem. 48:1314-1320 (2002); Molina et al. Clin. Cancer Res. 8:347-353 (2002); U.S. Pat. No. 4,933,294 issued Jun. 12, 1990; WO91/05264 published Apr.18, 1991; U.S. Pat. No. 5,401,638 issued March 28, 1995; or Sias et al. J. Immunol. Methods 132: 73-80 (1990).

Herein, “elevated shed HER2 serum level” refers to an amount of shed HER2 or HER2 ECD in the serum of a mammal (e.g. human) that exceeds the amount present in the serum a normal mammal (e.g. human). Elevated HER2 ECD serum levels may correlate with a poor prognosis and decreased responsiveness to endocrine therapy and chemotherapy in patients with advanced breast cancer.

The expression “p95 HER2” herein refers to NH2-terminal truncated HER2 protein. Generally, p95 is a membrane-bound stub fragment which may arise from cleavage of full length HER2 by a protease or sheddase (Yuan et al. Protein Expression and Purification 29: 217-222 (2003)). p95 may have a Mᵣ of about 95,000 and may be phosphorylated (Molina et al. Cancer Research 47444749 (2001)). p95 has been found in some breast cancer samples (Christianson et al. Cancer Res. 15:5123-5129 (1998)).

By “elevated p95 level” is meant a level of p95 in a cancer cell that exceeds the normal level, for instance the level in a normal or non-cancerous cell of the same tissue type as the cancer cell. Such elevated p95 level may result in constitutive signaling, and nodal metastasis (Molina et al. Clin. Cancer Research 8:347-353 (2002); Christianson et al. Cancer Res. 15:5123-5129 (1998)).

By “evaluating” a marker, such as MMP-15, is intended a diagnostic and/or prognostic analysis thereof, including an analysis of the presence or absence of that marker, measurement of the amount thereof, and/or an analysis of activity thereof (e.g. increased activity).

A cancer patient with “a poor clinical outcome” is one with a poor prognosis, who is less likely to respond to cancer therapy, such as chemotherapy or therapy with a HER2 antibody, such as trastuzumab. The clinical outcome can be measured by standard means, such as survival, including disease free survival, etc.

“ErbB3” and “HER3” refer to the receptor polypeptide as disclosed, for example, in U.S. Pat. Nos. 5,183,884 and 5,480,968 as well as Kraus et al. PNAS (USA) 86:9193-9197 (1989), including variant forms thereof.

The terms “ErbB4” and “HER4” herein refer to the receptor polypeptide as disclosed, for example, in EP Pat Appln No 599,274; Plowman et al., Proc. Natl. Acad Sci. USA, 90:1746-1750 (1993); and Plowman et al., Nature, 366:473475 (1993), including variant forms thereof, such as the isoforms disclosed in WO99/19488, published Apr. 22, 1999.

By “HER ligand” is meant a polypeptide which binds to and/or activates a HER receptor. The HER ligand of particular interest herein is a native sequence human HER ligand such as epidermal growth factor (EGF) (Savage et al., J. Biol. Chem. 247:7612-7621 (1972)); transforming growth factor alpha (TGF-α) (Marquardt et al., Science 223:1079-1082 (1984)); amphiregulin also known as schwanoma or keratinocyte autocrine growth factor (Shoyab et al. Science 243:1074-1076 (1989); Kimura et al. Nature 348:257-260 (1990); and Cook et al. Mol. Cell. Biol. 11:2547-2557 (1991)); betacellulin (Shing et al., Science 259:1604-1607 (1993); and Sasada et al. Biochem. Biophys. Res. Commun. 190:1173 (1993)); heparin-binding epidermal growth factor (HB-EGF) (Higashiyama et al., Science 251:936-939 (1991)); epiregulin (Toyoda et al., J. Biol. Chem. 270:7495-7500 (1995); and Komurasaki et al. Oncogene 15:2841-2848 (1997)); a heregulin (see below); neuregulin-2 (NRG-2) (Carraway et al., Nature 387:512-516 (1997)); neuregulin-3 (NRG-3) (Zhang et al., Proc. Natl. Acad Sci. 94:9562-9567 (1997)); neuregulin-4 (NRG4) (Harari et al. Oncogene 18:2681-89 (1999)); and cripto (CR-1) (Kannan et al. J. Biol. Chem. 272(6):3330-3335 (1997)). HER ligands which bind EGFR include EGF, TGF-α, amphiregulin, betacellulin, HB-EGF and epiregulin. HER ligands which bind HER3 include heregulins. HER ligands capable of binding HER4 include betacellulin, epiregulin, HB-EGF, NRG-2, NRG-3, NRG4, and heregulins.

“Heregulin” (HRG) when used herein refers to a polypeptide encoded by the heregulin gene product as disclosed in U.S. Pat. No. 5,641,869, or Marchionni et al., Nature, 362:312-318 (1993). Examples of heregulins include heregulin-α, heregulin-β1, heregulin-β2 and heregulin-β3 (Holmes et al., Science, 256:1205-1210 (1992); and U.S. Pat. No. 5,641,869); neu differentiation factor (NDF) (Peles et al. Cell 69: 205-216 (1992)); acetylcholine receptor-inducing activity (ARIA) (Falls et al. Cell 72:801-815 (1993)); glial growth factors (GGFs) (Marchionni et al., Nature, 362:312-318 (1993)); sensory and motor neuron derived factor (SMDF) (Ho et al. J. Biol. Chem. 270:14523-14532 (1995)); γ-heregulin (Schaefer et al. Oncogene 15:1385-1394 (1997)).

A “HER dimer” herein is a noncovalently associated dimer comprising at least two HER receptors. Such complexes may form when a cell expressing two or more HER receptors is exposed to an HER ligand and can be isolated by immunoprecipitation and analyzed by SDS-PAGE as described in Sliwkowski et al., J. Biol. Chem., 269(20):14661-14665 (1994), for example. Examples of such HER dimers include EGFR-HER2, HER2-HER3 and HER3-HER4 heterodimers. Moreover, the HER dimer may comprise two or more HER2 receptors combined with a different HER receptor, such as HER3, HER4 or EGFR. Other proteins, such as a cytokine receptor subunit (e.g. gp130) may be associated with the dimer.

A cell, cancer, or biological sample which “displays HER expression, amplification, or activation” is one which, in a diagnostic test, expresses (including overexpresses) HER, has amplified HER gene, and/or otherwise demonstrates activation or phosphorylation of HER receptor(s). Such activation can be determined directly (e.g. by measuring HER phosphorylation) or indirectly (e.g. by gene expression profiling or by detecting HER heterodimers).

A cancer or tumor cell with “HER2 receptor overexpression or amplification” is one which has significantly higher levels of a HER2 protein or gene compared to a noncancerous cell of the same tissue type. Such overexpression may be caused by gene amplification or by increased transcription or translation. HER2 overexpression or amplification may be determined in a diagnostic or prognostic assay by evaluating increased levels of the HER2 protein present on the surface of a cell (e.g. via an immunohistochemistry assay; IHC). Alternatively, or additionally, one may measure levels of HER2 nucleic acid in the cell, e.g. via fluorescent in situ hybridization (FISH; see WO98/45479 published October 1998), southern blotting, or polymerase chain reaction (PCR) techniques, such as quantitative real time PCR (qRT-PCR). One may also study HER2 overexpression or amplification by measuring shed HER2 in a biological fluid such as serum (see, e.g., U.S. Pat. No. 4,933,294 issued Jun. 12, 1990; WO91/05264 published Apr.18, 1991; U.S. Pat. No. 5,401,638 issued Mar. 28, 1995; and Sias et al. J. Immunol. Methods 132: 73-80 (1990)). Aside from the above assays, various in vivo assays are available to the skilled practitioner. For example, one may expose cells within the body of the patient to an antibody which is optionally labeled with a detectable label, e.g. a radioactive isotope, and binding of the antibody to cells in the patient can be evaluated, e.g. by external scanning for radioactivity or by analyzing a biopsy taken from a patient previously exposed to the antibody.

Conversely, a cancer or tumor cell which “does not overexpress or amplify HER receptor” is one which does not have higher than normal levels of HER receptor protein or gene compared to a noncancerous cell of the same tissue type. Antibodies that inhibit HER dimerization, such as pertuzumab, may be used to treat cancer which does not overexpress or amplify HER2 receptor.

A “HER inhibitor” is an agent which interferes with HER activation or function. Examples of HER inhibitors include HER antibodies (e.g EGFR, HER2, HER3, or HER4 antibodies); HER dimerization inhibitors; EGFR-targeted drugs; small molecule HER antagonists; HER tyrosine kinase inhibitors; HER2 and EGFR dual tyrosine kinase inhibitors such as lapatinib/GW572016; antisense molecules (see, for example, WO2004/87207); and/or agents that bind to, or interfere with function of, downstream signaling molecules, such as MAPK or Akt. Preferably, the HER inhibitor is an antibody or small molecule which binds to a HER receptor. Specific examples of HER inhibitors include trastuzumab, pertuzumab, cetuximab, ABX-EGF, EMD7200, gefitinib, erlotinib, CP724714, C11033, GW572016, IMC-11F8, and TAK165.

A “HER dimerization inhibitor” is an agent which inhibits formation of a HER dimer. Preferably, the HER dimerization inhibitor is. an antibody, for example an antibody which binds to HER2 at the heterodimeric binding site thereof. The most preferred dimerization inhibitor herein is pertuzumab or monoclonal antibody 2C4 (MAb 2C4). Other examples of HER dimerization inhibitors include antibodies which bind to EGFR and inhibit dimerization thereof with one or more other HER receptors (for example EGFR monoclonal antibody 806, MAb 806, which binds to activated or “untethered” EGFR; see Johns et al., J. Biol. Chem. 279(29):30375-30384 (2004)); antibodies which bind to HER3 and inhibit dimerization thereof with one or more other HER receptors; antibodies which bind to HER4 and inhibit dimerization thereof with one or more other HER receptors; peptide dimerization inhibitors (U.S. Pat. No. 6,417,168); antisense dimerization inhibitors; etc.

A “HER antibody” is an antibody that binds to a HER receptor. Optionally, the HER antibody further interferes with HER activation or function. Preferably, the HER antibody binds to the HER2 receptor. HER2 antibodies of particular interest herein are trastuzumab and pertuzumab. Examples of EGFR antibodies include cetuximab, ABX0303, EMD7200 and IMC-11F5.

“HER activation” refers to activation, or phosphorylation, of any one or more HER receptors. Generally, HER activation results in signal transduction (e.g. that caused by an intracellular kinase domain of a HER receptor phosphorylating tyrosine residues in the HER receptor or a substrate polypeptide). HER activation may be mediated by HER ligand binding to a HER dimer comprising the HER receptor of interest. HER ligand binding to a HER dimer may activate a kinase domain of one or more of the HER receptors in the dimer and thereby results in phosphorylation of tyrosine residues in one or more of the HER receptors and/or phosphorylation of tyrosine residues in additional substrate polypeptides(s), such as Akt or MAPK intracellular kinases.

“Phosphorylation” refers to the addition of one or more phosphate group(s) to a protein, such as a HER receptor, or substrate thereof.

An antibody which “inhibits HER dimerization” is an antibody which inhibits, or interferes with, formation of a HER dimer. Preferably, such an antibody binds to HER2 at the heterodimeric binding site thereof. The most preferred dimerization inhibiting antibody herein is pertuzumab or MAb 2C4. Other examples of antibodies which inhibit HER dimerization include antibodies which bind to EGFR and inhibit dimerization thereof with one or more other HER receptors (for example EGFR monoclonal antibody 806, MAb 806, which binds to activated or “untethered” EGFR; see Johns et al., J. Biol. Chem. 279(29):30375-30384 (2004)); antibodies which bind to HER3 and inhibit dimerization thereof with one or more other HER receptors; and antibodies which bind to HER4 and inhibit dimerization thereof with one or more other HER receptors.

A “heterodimeric binding site” on HER2, refers to a region in the extracellular domain of HER2 that contacts, or interfaces with, a region in the extracellular domain of EGFR, HER3 or HER4 upon formation of a dimer therewith. The region is found in Domain II of HER2. Franklin et al. Cancer Cell 5:317-328 (2004).

The HER2 antibody may be one which, like trastuzumab, “inhibits HER2 ectodomain cleavage” (Molina et al. Cancer Res. 61:47444749(2001)) or may be one which, like pertuzumab, does not significantly inhibit HER2 ectodomain cleavage.

A HER2 antibody that “binds to a heterodimeric binding site” of HER2, binds to residues in domain II (and optionally also binds to residues in other of the domains of the HER2 extracellular domain, such as domains I and III), and can sterically hinder, at least to some extent, formation of a HER2-EGFR, HER2-HER3, or HER2-HER4 heterodimer. Franklin et al. Cancer Cell 5:317-328 (2004) characterize the HER2-pertuzumab crystal structure, deposited with the RCSB Protein Data Bank (ID Code IS78), illustrating an exemplary antibody that binds to the heterodimeric binding site of HER2.

An antibody that “binds to domain II ” of HER2 binds to residues in domain II and optionally residues in other domain(s) of HER2, such as domains I and III. Preferably the antibody that binds to domain II binds to the junction between domains I, II and III of HER2.

A “native sequence” polypeptide is one which has the same amino acid sequence as a polypeptide (e.g., HER receptor or HER ligand) derived from nature. Such native sequence polypeptides can be isolated from nature or can be produced by recombinant or synthetic means. Thus, a native sequence polypeptide can have the amino acid sequence of naturally occurring human polypeptide, murine polypeptide, or polypeptide from any other mammalian species.

The term “antibody” herein is used in the broadest sense and specifically covers intact monoclonal antibodies, polyclonal antibodies, multispecific antibodies (e.g. bispecific antibodies) formed from at least two intact antibodies, and antibody fragments, so long as they exhibit the desired biological activity.

The term “monoclonal antibody” as used herein refers to an antibody from a population of substantially homogeneous antibodies, i e., the individual antibodies comprising the population are identical and/or bind the same epitope(s), except for possible variants that may arise during production of the monoclonal antibody, such variants generally being present in minor amounts. Such monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones or recombinant DNA clones. It should be understood that the selected target binding sequence can be fuirther altered, for example, to improve affinity for the target, to humanize the target binding sequence, to improve its production in cell culture, to reduce its immunogenicity in vivo, to create a multispecific antibody, etc., and that an antibody comprising the altered target binding sequence is also a monoclonal antibody of this invention. In contrast to polyclonal antibody preparations which typically include different antibodies directed against different determinants (epitopes), each monoclonal antibody of a monoclonal antibody preparation is directed against a single determinant on an antigen. In addition to their specificity, the monoclonal antibody preparations are advantageous in that they are typically uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, for example, the hybridoma method (e.g., Kohler et al., Nature, 256:495 (1975); Harlow et al., Antibodies: A Laboratory Manual, (Cold Spring Harbor Laboratory Press, 2nd ed. 1988); Hammerling et al., in: Monoclonal Antibodies and T-Cell Hybridomas 563-681, (Elsevier, N.Y., 1981)), recombinant DNA methods (see, e.g., U.S. Pat. No. 4,816,567), phage display technologies (see, e.g., Clackson et al., Nature, 352:624-628 (1991); Marks et al., J. Mol. Biol., 222:581-597 (1991); Sidhu et al., J. Mol. Biol. 338(2):299-310 (2004); Lee et al., J.Mol.Biol.340(5):1073-1093 (2004); Fellouse, Proc. Nat. Acad. Sci. USA 101(34):12467-12472 (2004); and Lee et al. J. Immunol. Methods 284(1-2):119-132 (2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad Sci. USA, 90:2551 (1993); Jakobovits et al., Nature, 362:255-258 (1993); Bruggemann et al., Year in Immuno., 7:33 (1993); U.S. Pat. Nos. 5,545,806; 5,569,825; 5,591,669 (all of GenPharm); U.S. Pat. No. 5,545,807; WO 1997/17852; U.S. Pat. Nos. 5,545,807; 5,545,806; 5,569,825; 5,625,126; 5,633,425; and 5,661,016; Marks et al., Bio/Technology, 10: 779-783 (1992); Lonberg et al., Nature, 368: 856-859 (1994); Morrison, Nature, 368: 812-813 (1994); Fishwild et al., Nature Biotechnology, 14: 845-851 (1996); Neuberger, Nature Biotechnology, 14: 826 (1996); and Lonberg and Huszar, Intern. Rev. Immunol., 13: 65-93 (1995).

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA, 81:6851-6855 (1984)). Chimeric antibodies of interest herein include “primatized” antibodies comprising variable domain antigen-binding sequences derived from a non-human primate (e.g. Old World Monkey, Ape etc) and human constant region sequences.

“Antibody fragments” comprise a portion of an intact antibody, preferably comprising the antigen binding region thereof. Examples of antibody fragments include Fab, Fab′, F(ab′)₂, and Fv fragments; diabodies; linear antibodies; single-chain antibody molecules; and multispecific antibodies formed from antibody fragment(s).

An “intact antibody” herein is one which comprises two antigen binding regions, and an Fc region. Preferably, the intact antibody has one or more effector functions.

Depending on the amino acid sequence of the constant domain of their heavy chains, intact antibodies can be assigned to different “classes”. There are five major classes of intact antibodies: IgA, IgD, IgE, IgG, and IgM, and several of these may be further divided into “subclasses” (isotypes), e.g., IgG1, IgG2, IgG3, IgG4, IgA, and IgA2. The heavy-chain constant domains that correspond to the different classes of antibodies are called α, δ, ε, γ, and μ, respectively. The subunit structures and three-dimensional configurations of different classes of immunoglobulins are well known.

Antibody “effector functions” refer to those biological activities attributable to an Fc region (a native sequence Fc region or amino acid sequence variant Fc region) of an antibody. Examples of antibody effector functions include C1q binding; complement dependent cytotoxicity; Fc receptor binding; antibody-dependent cell-mediated cytotoxicity (ADCC); phagocytosis; down regulation of cell surface receptors (e.g. B cell receptor; BCR), etc.

“Antibody-dependent cell-mediated cytotoxicity” and “ADCC” refer to a cell-mediated reaction in which nonspecific cytotoxic cells that express Fc receptors (FcRs) (e.g. Natural Killer (NK) cells, neutrophils, and macrophages) recognize bound antibody on a target cell and subsequently cause lysis of the target cell. The primary cells for mediating ADCC, NK cells, express FcγRIII only, whereas monocytes express FcγRI, FcγRII and FcγRIII. FcR expression on hematopoietic cells in summarized is Table 3 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. PNAS (USA) 95:652-656 (1998).

“Human effector cells” are leukocytes which express one or more FcRs and perform effector ftunctions. Preferably, the cells express at least FcγRIII and perform ADCC effector function. Examples of human leukocytes which mediate ADCC include peripheral blood mononuclear cells (PBMC), natural killer (NK) cells, monocytes, cytotoxic T cells and neutrophils; with PBMCs and NK cells being preferred. The effector cells may be isolated from a native source thereof, e.g. from blood or PBMCs as described herein.

The terms “Fc receptor” or “FcR” are used to describe a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain (see review M. in Daëron, Annu. Rev. Immunol. 15:203-234 (1997)). FcRs are reviewed in Ravetch and Kinet, Annu. Rev. Immunol 9:457-92 (1991); Capel et al., Immunomethods 4:25-34 (1994); and de Haas et al., J. Lab. Clin. Med. 126:33041 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976) and Kim et al, J. Immunol. 24:249 (1994)), and regulates homeostasis of immunoglobulins.

“Complement dependent cytotoxicity” or “CDC” refers to the ability of a molecule to lyse a target in the presence of complement. The complement activation pathway is initiated by the binding of the first component of the complement system (C1q) to a molecule (e.g. an antibody) complexed with a cognate antigen. To assess complement activation, a CDC assay, e.g as described in Gazzano-Santoro et al., J. Immunol. Methods 202:163 (1996), may be performed.

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end. The constant domain of the light chain is aligned with the first constant domain of the heavy chain, and the light-chain variable domain is aligned with the variable domain of the heavy chain. Particular amino acid residues are believed to form an interface between the light chain and heavy chain variable domains.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions both in the light chain and the heavy chain variable domains. The more highly conserved portions of variable domains are called the framework regions (FRs). The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC).

The term “hypervariable region” when used herein refers to the amino acid residues of an antibody which are responsible for antigen-binding. The hypervariable region generally comprises amino acid residues from a “complementary determining region” or “CDR” (e.g residues 24-34 (L1), 50-56 (L2) and 89-97 (L3) in the light chain variable domain and 31-35 (HI), 50-65 (H2) and 95-102 (H3) in the heavy chain variable domain; Kabat et al, Sequences of Proteins of Immunological Interest, 5th Ed. Public Health Service, National Institutes of Health, Bethesda, Md. (1991)) and/or those residues from a “hypervariable loop” (e.g. residues 26-32 (L1), 50-52 (L2) and 91-96 (L3) in the light chain variable domain and 26-32 (H1), 53-55 (H2) and 96-101 (H3) in the heavy chain variable domain; Chothia and Lesk J. Mol. Biol. 196:901-917 (1987)). “Framework Region” or “FR” residues are those variable domain residues other than the hypervariable region residues as herein defined.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-binding sites and is still capable of cross-linking antigen.

“Fv” is the minimum antibody fragment which contains a complete antigen-recognition and antigen-binding site. This region consists of a dimer of one heavy chain and one light chain variable domain in tight, non-covalent association. It is in this configuration that the three hypervariable regions of each variable domain interact to defme an antigen-binding site on the surface of the V_{H}-V_{L} dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site.

The Fab fragment also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear at least one free thiol group. F(ab′)₂ antibody fragments originally were produced as pairs of Fab′ fragments which have hinge cysteines between them. Other chemical couplings of antibody fragments are also known.

The “light chains” of antibodies from any vertebrate species can be assigned to one of two clearly distinct types, called kappa (κ) and lambda (λ), based on the amino acid sequences of their constant domains.

“Single-chain Fv” or “scFv” antibody fragments comprise the V_{H} and V_{L} domains of antibody, wherein these domains are present in a single polypeptide chain. Preferably, the Fv polypeptide further comprises a polypeptide linker between the V_{H} and V_{L} domains which enables the scFv to form the desired structure for antigen binding. For a review of scFv see Plückthun in The Pharmacology of Monoclonal Antibodies, vol. 113, Rosenburg and Moore eds., Springer-Verlag, New York, pp. 269-315 (1994). HER2 antibody scFv fragments are described in WO93/16185; U.S. Pat. No. 5,571,894; and U.S. Pat. No. 5,587,458.

The term “diabodies” refers to small antibody fragments with two antigen-binding sites, which fragments comprise a variable heavy domain (V_{H}) connected to a variable light domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al, Proc. Natl. Acad. Sci USA, 90:6444-6448 (1993).

“Humanized” forms of non-human (e.g., rodent) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al, Nature 332:323-329 (1988); and Presta, Curr. Op. Struct. Biol. 2:593-596 (1992).

Humanized HER2 antibodies include huMAb4D5-1, huMAb4D5-2, huMAb4D5-3, huMAb4D5-4, huMAb4D5-5, huMAb4D5-6, huMAb4D5-7 and huMAb4D5-8 or trastuzumab (HERCEPTIN®) as described in Table 3 of U.S. Pat. No. 5,821,337 expressly incorporated herein by reference; humanized 520C9 (WO93/21319); and humanized 2C4 antibodies such as pertuzumab as described herein.

For the purposes herein, “trastuzumab,” “HERCEPTIN®,” and “huMAb4D5-8” refer to an antibody comprising the light and heavy chain amino acid sequences in SEQ ID NOS. 5 and 6, respectively.

Herein, “pertuzumab” and “OMNITARG™” refer to an antibody comprising the light and heavy chain amino acid sequences in SEQ ID NOS. 7 and 8, respectively.

A “naked antibody” is an antibody that is not conjugated to a heterologous molecule, such as a cytotoxic moiety or radiolabel.

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

An “affinity matured” antibody is one with one or more alterations in one or more hypervariable regions thereof which result an improvement in the affinity of the antibody for antigen, compared to a parent antibody which does not possess those alteration(s). Preferred affinity matured antibodies will have nanomolar or even picomolar affinities for the target antigen. Affinity matured antibodies are produced by procedures known in the art. Marks et al. Bio/Technology 10:779-783 (1992) describes affinity maturation by VH and VL domain shuffling. Random mutagenesis of CDR and/or framework residues is described by: Barbas et al. Proc Nat. Acad Sci, USA 91:3809-3813 (1994); Schier et al. Gene 169:147-155 (1995); Yelton et al. J. Immunol 155:1994-2004 (1995); Jackson et al., J. Immunol. 154(7):3310-9 (1995); and Hawkins et al, J. Mol. Biol. 226:889-896 (1992).

The term “main species antibody” herein refers to the antibody structure in a composition which is the quantitatively predominant antibody molecule in the composition.

An “amino acid sequence variant” antibody herein is an antibody with an amino acid sequence which differs from a main species antibody. Ordinarily, amino acid sequence variants will possess at least about 70% homology with the main species antibody, and preferably, they will be at least about 80%, more preferably at least about 90% homologous with the main species antibody. The amino acid sequence variants possess substitutions, deletions, and/or additions at certain positions within or adjacent to the amino acid sequence of the main species antibody.

A “glycosylation variant” antibody herein is an antibody with one or more carbohydrate moeities attached thereto which differ from one or more carbohydate moieties attached to a main species antibody.

A “deamidated” antibody is one in which one or more asparagine residues thereof has been derivitized, e.g. to an aspartic acid, a succinimide, or an iso-aspartic acid.

The terms “cancer” and “cancerous” refer to or describe the physiological condition in mammals that is typically characterized by unregulated cell growth. Examples of cancer include, but are not limited to, carcinoma, lymphoma, blastoma (including medulloblastoma and retinoblastoma), sarcoma (including liposarcoma and synovial cell sarcoma), neuroendocrine tumors (including carcinoid tumors, gastrinoma, and islet cell cancer), mesothelioma, schwannoma (including acoustic neuroma), meningioma, adenocarcinoma, melanoma, and leukemia or lymphoid malignancies. More particular examples of such cancers include squamous cell cancer (e.g. epithelial squamous cell cancer), lung cancer including small-cell lung cancer (SCLC), non-small cell lung cancer (NSCLC), adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer (including metastatic breast cancer), colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, testicular cancer, esophagael cancer, tumors of the biliary tract, as well as head and neck cancer.

A mammal with a “HER inhibitor-resistant cancer” is one who has progressed while receiving HER inhibitor-based therapy (i.e. the patient is “HER inhibitor refractory”), or the mammal has progressed within 12 months (for instance, within 6 months) after completing a HER inhibitor-based therapy regimen. The HER inhibitor-based therapy includes therapy with naked or conjugated HER inhibitor, where the HER inhibitor is administered as a single-agent, or in combination with other anti-tumor drug(s). The HER inhibitor may be trastuzumab, pertuzumab, cetuximab, ABX-EGF, EMD7200, gefitinib, erlotinib, CP724714, CI1033, GW572016, IMC-11F8, or TAK165, but preferably is trastuzumab.

A “mammal” for purposes of treatment refers to any animal classified as a mammal, including humans, domestic and farm animals, and zoo, sports, or pet animals, such as dogs, horses, cats, cows, etc. Preferably, the mammal is human.

A “tumor sample” herein is a sample derived from, or comprising tumor cells from, a patient's tumor. Examples of tumor samples herein include, but are not limited to, tumor biopsies, circulating tumor cells, circulating plasma proteins, ascitic fluid, primary cell cultures or cell lines derived from tumors or exhibiting tumor-like properties, as well as preserved tumor samples, such as formalin-fixed, paraffin-embedded tumor samples or frozen tumor samples.

A “fixed” tumor sample is one which has been histologically preserved using a fixative.

A “formalin-fixed” tumor sample is one which has been preserved using formaldehyde as the fixative.

An “embedded” tumor sample is one surrounded by a firm and generally hard medium such as paraffm, wax, celloidin, or a resin. Embedding makes possible the cutting of thin sections for microscopic examination or for generation of tissue microarrays (TMAs).

A “paraffin-embedded” tumor sample is one surrounded by a purified mixture of solid hydrocarbons derived from petroleum.

Herein, a “frozen” tumor sample refers to a tumor sample which is, or has been, frozen.

A “growth inhibitory agent” when used herein refers to a compound or composition which inhibits growth of a cell, especially a HER expressing cancer cell either in vitro or in vivo. Thus, the growth inhibitory agent may be one which significantly reduces the percentage of HER expressing cells in S phase. Examples of growth inhibitory agents include agents that block cell cycle progression (at a place other than S phase), such as agents that induce G1 arrest and M-phase arrest. Classical M-phase blockers include the vincas (vincristine and vinblastine), taxanes, and topo II inhibitors such as doxorubicin, epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in The Molecular Basis of Cancer, Mendelsohn and Israel, eds., Chapter 1, entitled “Cell cycle regulation, oncogenes, and antineoplastic drugs” by Murakami et al. (W B Saunders: Philadelphia, 1995), especially p. 13.

Examples of “growth inhibitory” antibodies are those which bind to HER2 and inhibit the growth of cancer cells overexpressing HER2. Preferred growth inhibitory HER2 antibodies inhibit growth of SK-BR-3 breast tumor cells in cell culture by greater than 20%, and preferably greater than 50% (e.g. from about 50% to about 100%) at an antibody concentration of about 0.5 to 30 μg/ml, where the growth inhibition is determined six days after exposure of the SK-BR-3 cells to the antibody (see U.S. Pat. No. 5,677,171 issued Oct. 14, 1997). The SK-BR-3 cell growth inhibition assay is described in more detail in that patent and hereinbelow. The preferred growth inhibitory antibody is a humanized variant of murine monoclonal antibody 4D5, e.g., trastuzumab.

An antibody which “induces apoptosis” is one which induces programmed cell death as determined by binding of annexin V, fragmentation of DNA, cell shrinkage, dilation of endoplasmic reticulum, cell fragmentation, and/or formation of membrane vesicles (called apoptotic bodies). The cell is usually one which overexpresses the HER2 receptor. Preferably the cell is a tumor cell, e.g. a breast, ovarian, stomach, endometrial, salivary gland, lung, kidney, colon, thyroid, pancreatic or bladder cell. In vitro, the cell may be a SK-BR-3, BT474, Calu 3 cell, MDA-MB-453, MDA-MB-361 or SKOV3 cell. Various methods are available for evaluating the cellular events associated with apoptosis. For example, phosphatidyl serine (PS) translocation can be measured by annexin binding; DNA fragmentation can be evaluated through DNA laddering; and nuclear/chromatin condensation along with DNA fragmentation can be evaluated by any increase in hypodiploid cells. Preferably, the antibody which induces apoptosis is one which results in about 2 to 50 fold, preferably about 5 to 50 fold, and most preferably about 10 to 50 fold, induction of annexin binding relative to untreated cell in an annexin binding assay using BT474 cells (see below). Examples of HER2 antibodies that induce apoptosis are 7C2 and 7F3.

The “epitope 2C4” is the region in the extracellular domain of HER2 to which the antibody 2C4 binds. In order to screen for antibodies which bind to the 2C4 epitope, a routine cross-blocking assay such as that described in Antibodies, A Laboratory Manual, Cold Spring Harbor Laboratory, Ed Harlow and David Lane (1988), can be performed. Preferably the antibody blocks 2C4's binding to HER2 by about 50% or more. Alternatively, epitope mapping can be performed to assess whether the antibody binds to the 2C4 epitope of HER2. Epitope 2C4 comprises residues from Domain II in the extracellular domain of HER2. 2C4 and pertuzumab binds to the extracellular domain of HER2 at the junction of domains I, II and III. Franklin et al. Cancer Cell 5:317-328 (2004).

The “epitope 4D5” is the region in the extracellular domain of HER2 to which the antibody 4D5 (ATCC CRL 10463) and trastuzumab bind. This epitope is close to the transmembrane domain of HER2, and within Domain IV of HER2. To screen for antibodies which bind to the 4D5 epitope, a routine cross-blocking assay such as that described in Antibodies, A Laboratory Manual, Cold Spring Harbor Laboratory, Ed Harlow and David Lane (1988), can be performed. Alternatively, epitope mapping can be performed to assess whether the antibody binds to the 4D5 epitope of HER2 (e.g. any one or more residues in the region from about residue 529 to about residue 625, inclusive of the HER2 ECD, residue numbering including signal peptide).

The “epitope 7C2/7F3” is the region at the N terminus, within Domain I, of the extracellular domain of HER2 to which the 7C2 and/or 7F3 antibodies (each deposited with the ATCC, see below) bind. To screen for antibodies which bind to the 7C2/7F3 epitope, a routine cross-blocking assay such as that described in Antibodies, A Laboratory Manual, Cold Spring Harbor Laboratory, Ed Harlow and David Lane (1988), can be performed. Alternatively, epitope mapping can be performed to establish whether the antibody binds to the 7C2/7F3 epitope on HER2 (e.g. any one or more of residues in the region from about residue 22 to about residue 53 of the HER2 ECD, residue numbering including signal peptide).

“Treatment” refers to both therapeutic treatment and prophylactic or preventative measures. Those in need of treatment include those already with the disease as well as those in which the disease is to be prevented. Hence, the patient to be treated herein may have been diagnosed as having the disease or may be predisposed or susceptible to the disease.

The term “effective amount” refers to an amount of a drug effective to treat cancer in the patient. The effective amount of the drug may reduce the number of cancer cells; reduce the tumor size; inhibit (ie., slow to some extent and preferably stop) cancer cell infiltration into peripheral organs; inhibit (ie., slow to some extent and preferably stop) tumor metastasis; inhibit, to some extent, tumor growth; and/or relieve to some extent one or more of the symptoms associated with the cancer. To the extent the drug may prevent growth and/or kill existing cancer cells, it may be cytostatic and/or cytotoxic. The effective amount may extend progression free survival, result in an objective response (including a partial response, PR, or complete response, CR), increase overall survival time, and/or improve one or more symptoms of cancer.

By “complete response” or “complete remission” is intended the disappearance of all signs of cancer in response to treatment. This does not always mean the cancer has been cured.

“Partial response” refers to a decrease in the size of one or more tumors or lesions, or in the extent of cancer in the body, in response to treatment.

The term “cytotoxic agent” as used herein refers to a substance that inhibits or prevents the fuinction of cells and/or causes destruction of cells. The term is intended to include radioactive isotopes (e.g. At²¹¹, I¹³¹, I¹²⁵, Y⁹⁰, Re¹⁸⁶, Re¹⁸⁸, Sm¹⁵³, Bi²¹², P³² and radioactive isotopes of Lu), chemotherapeutic agents, and toxins such as small molecule toxins or enzymatically active toxins of bacterial, fungal, plant or animal origin, including fragments and/or variants thereof.

A “chemotherapeutic agent” is a chemical compound useful in the treatment of cancer. Examples of chemotherapeutic agents include alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; TLK 286 (TELCYTA™); acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; bisphosphonates, such as clodronate; antibiotics such as the enediyne antibiotics (e. g., calicheamicin, especially calicheamicin gammall and calicheamicin omegaI1 (see, e.g., Agnew, Chem Intl. Ed. Engl., 33: 183-186 (1994)) and anthracyclines such as annamycin, AD 32, alcarubicin, daunorubicin, dexrazoxane, DX-52-1, epirubicin, GPX-100, idarubicin, KRN5500, menogaril, dynemic in, including dynemicin A, an esperamicin, neocarzinostatin chromophore and related chromoprotein enediyne antiobiotic chromophores, aclacinomysins, actinomycin, authramycin, azaserine, bleomycins, cactinomycin, carabicin, carminomycin, carzinophilin, chromomycinis, dactinomycin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin, liposomal doxorubicin, and deoxydoxorubicin), esorubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, and zorubicin; folic acid analogues such as denopterin, pteropterin, and trimetrexate; purine analogs such as fludarabine, 6-mercaptopurine, thiamiprine, and thioguanine; pyrimidine analogs such as ancitabine, azacitidine, 6-azauridine, carmoftir, cytarabine, dideoxyuridine, doxifluridine, enocitabine, and floxuridine; androgens such as calusterone, dromostanolone propionate, epitiostanol, mepitiostane, and testolactone; anti-adrenals such as aminoglutethimide, mitotane, and trilostane; folic acid replenisher such as folinic acid (leucovorin); aceglatone; anti-folate anti-neoplastic agents such as ALIMTA®, LY231514 pemetrexed, dihydrofolate reductase inhibitors such as methotrexate, anti-metabolites such as 5-fluorouracil (5-FU) and its prodrugs such as UFT, S-1 and capecitabine, and thymidylate synthase inhibitors and glycinamide ribonucleotide formyltransferase inhibitors such as raltitrexed (TOMUDEX™, TDX); inhibitors of dihydropyrimidine dehydrogenase such as eniluracil; aldophosphamide glycoside; aminolevulinic acid; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine (ELDISINE®, FILDESIN®) ; dacarbazine; mannomustine; mitobronitol; mitolactol; pipobroman; gacytosine; arabinoside (“Ara-C”); cyclophosphamide; thiotepa; taxoids and taxanes, e.g., TAXOL® paclitaxel (Bristol-Myers Squibb Oncology, Princeton, N.J.), ABRAXANE™ Cremophor-free, albumin-engineered nanoparticle formulation of paclitaxel (American Pharmaceutical Partners, Schaumberg, Ill.), and TAXOTERE® docetaxel (Rhône-Poulenc Rorer, Antony, France); chloranbucil; antimetabolite chemotherapeutic agent such as gemcitabine (GEMZAR®), 5-fluorouracil (5-FU), capecitabine (XELODA™), 6-mercaptopurine, methotrexate, 6-thioguanine, pemetrexed, raltitrexed, arabinosylcytosine ARA-C cytarabine (CYTOSAR-U®), dacarbazine (DTIC-DOME®), azocytosine, deoxycytosine, pyridmidene, fludarabine (FLUDARA®), cladrabine, and 2-deoxy-D-glucose; 6-thioguanine; mercaptopurine; platinum-based chemotherapeutic agent such as carboplatin, cisplatin, or oxaliplatinum; vinblastine (VELBAN®); etoposide (VP-16); ifosfamide; mitoxantrone; vincristine (ONCOVIN®); vinca alkaloid; vinorelbine (NAVELBINE®); novantrone; edatrexate; daunomycin; aminopterin; xeloda; ibandronate; topoisomerase inhibitor RFS 2000; difluorometlhylomithine (DMFO); retinoids such as retinoic acid; pharmaceutically acceptable salts, acids or derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATIN™) combined with 5-FU and leucovorin.

Also included in this definition are anti-hormonal agents that act to regulate or inhibit hormone action on tumors such as anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON® toremifene; aromatase inhibitors that inhibit the enzyme aromatase, which regulates estrogen production in the adrenal glands, such as, for example, 4(5)-imidazoles, aminoglutethimide, MEGASE® megestrol acetate, AROMASIN® exemestane, formestanie, fadrozole, RIVISOR® vorozole, FEMARA® letrozole, and ARIMIDEX® anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those that inhibit expression of genes in signaling pathways implicated in abherant cell proliferation, such as, for example, PKC-alpha, Raf, H-Ras, and epidermal growth factor receptor (EGF-R); vaccines such as gene therapy vaccines, for example, ALLOVECTIN® vaccine, LEUVECTIN® vaccine, and VAXID® vaccine; PROLEUKIN® rIL-2; LURTOTECAN® topoisomerase 1 inhibitor; ABARELIX® rmRH; and pharmaceutically acceptable salts, acids or derivatives of any of the above.

An “anti-angiogenic agent” refers to a compound which blocks, or interferes with to some degree, the development of blood vessels. The anti-angiogenic factor may, for instance, be a small molecule or antibody that binds to a growth factor or growth factor receptor involved in promoting angiogenesis. The preferred anti-angiogenic factor herein is an antibody that binds to vascular endothelial growth factor (VEGF), such as bevacizumab (AVASTINI®).

The term “cytokine” is a generic term for proteins released by one cell population which act on another cell as intercellular mediators. Examples of such cytokines are lymphokines, monokines, and traditional polypeptide hormones. Included among the cytokines are growth hormone such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-α and -β; mullerian-inhibiting substance; mouse gonadotropin-associated peptide; inhibin; activin; vascular endothelial growth factor; integrin; thrombopoietin (TPO); nerve growth factors such as NGF-β; platelet-growth factor; transforming growth factors (TGFs) such as TGF-α and TGF-β; insulin-like growth factor-I and -II; erythropoietin (EPO); osteoinductive factors; interferons such as interferon-α, -β, and -γ; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1a, IL-2, IL-3, IL-4, IL-5, IL-6, IL-7, IL-8, IL-9, IL-10, IL-11, IL-12; a tumor necrosis factor such as TNF-α or TNF-β; and other polypeptide factors including LIF and kit ligand (KL). As used herein, the term cytokine includes proteins from natural sources or from recombinant cell culture and biologically active equivalents of the native sequence cytokines.

As used herein, the term “EGFR-targeted drug” refers to a therapeutic agent that binds to EGFR and, optionally, inhibits EGFR activation. Examples of such agents include antibodies and small molecules that bind to EGFR. Examples of antibodies which bind to EGFR include MAb 579 (ATCC CRL HB 8506), MAb 455 (ATCC CRL HB8507), MAb 225 (ATCC CRL 8508), MAb 528 (ATCC CRL 8509) (see, U.S. Pat. No. 4,943, 533, Mendelsohn et al.) and variants thereof, such as chimerized 225 (C225 or Cetuximab; ERBUTIX®) and reshaped human 225 (H225) (see, WO 96/40210, Imclone Systems Inc.); IMC-11F8, a fully human, EGFR-targeted antibody (Imclone); antibodies that bind type II mutant EGFR (U.S. Pat. No. 5,212,290); humanized and chimeric antibodies that bind EGFR as described in U.S. Pat. No. 5,891,996; and human antibodies that bind EGFR, such as ABX-EGF (see WO98/50433, Abgenix); EMD 55900 (Stragliotto et al. Eur. J. Cancer 32A:636-640 (1996)); EMD7200 (matuzumab) a humanized EGFR antibody directed against EGFR that competes with both EGF and TGF-alpha for EGFR binding; and mAb 806 or humanized mAb 806 (Johns et al., J. Biol. Chem. 279(29):30375-30384 (2004)). The anti-EGFR antibody may be conjugated with a cytotoxic agent, thus generating an immunoconjugate (see, e.g., EP659,439A2, Merck Patent GmbH). Examples of small molecules that bind to EGFR include ZD1839 or Gefitinib (IRESSA™; Astra Zeneca); CP-358774 or Erlotinib (TARCEVA™; Genentech/OSI); and AG 1478, AG1571 (SU 5271; Sugen); EMD-7200.

A “tyrosine kinase inhibitor” is a molecule which inhibits tyrosine kinase activity of a tyrosine kinase such as a HER receptor. Examples of such inhibitors include the EGFR-targeted drugs noted in the preceding paragraph; small molecule HER2 tyrosine kinase inhibitor such as TAK165 available from Takeda; CP-724,714, an oral selective inhibitor of the ErbB2 receptor tyrosine kinase (Pfizer and OSI); dual-HER inhibitors such as EKB-569 (available from Wyeth) which preferentially binds EGFR but inhibits both HER2 and EGFR-overexpressing cells; GW572016 (available from Glaxo) an oral HER2 and EGFRtyrosine kinase inhibitor; PKI-166 (available from Novartis); pan-HER inhibitors such as canertinib (CI-1033; Pharmacia); Raf-1 inhibitors such as antisense agent ISIS-5132 available from ISIS Pharmaceuticals which inhibits Raf-1 signaling; non-HER targeted TK inhibitors such as Imatinib mesylate (Gleevac™) available from Glaxo; MAPK extracellular regulated kinase I inhibitor CI-1040 (available from Pharmacia); quinazolines, such as PD 153035,4-(3-chloroanilino) quinazoline; pyridopyrimidines; pyrimidopyrimidines; pyrrolopyrimidines, such as CGP 59326, CGP 60261 and CGP 62706; pyrazolopyrimidines, 4-(phenylamino)-7H-pyrrolo[2,3-d] pyrimidines; curcumin (diferuloyl methane, 4,5-bis (4-fluoroanilino)phthalimide); tyrphostines containing nitrothiophene moieties; PD-0183805 (Warner-Lamber); antisense molecules (e.g. those that bind to HER-encoding nucleic acid); quinoxalines (U.S. Pat. No. 5,804,396); tryphostins (U.S. Pat. No. 5,804,396); ZD6474 (Astra Zeneca); PTK-787 (Novartis/Schering AG); pan-HER inhibitors such as CI-1033 (Pfizer); Affinitac (ISIS 3521; Isis/Lilly); Imatinib mesylate (Gleevac; Novartis); PKI 166 (Novartis); GW2016 (Glaxo SmithKline); CI-1033 (Pfizer); EKB-569 (Wyeth); Semaxinib (Sugen); ZD6474 (AstraZeneca); PTK-787 (Novartis/Schering AG); INC-1C11 (Imclone); or as described in any of the following patent publications: U.S. Pat. No. 5,804,396; WO99/09016 (American Cyanimid); WO98/43960 (American Cyanamid); WO97/38983 (Warner Lambert); WO99/06378 (Warner Lambert); WO99/06396 (Warner Lambert); WO96/30347 (Pfizer, Inc); WO96/33978 (Zeneca); WO96/3397 (Zeneca); and WO96/33980 (Zeneca).

II. Inhibiting HER2 Shedding

The present application concerns a method for inhibiting HER2 shedding comprising treating, or exposing, a HER2 expressing cell with or to a matrix met alloprotease (MMP) antagonist in an amount effective to inhibit HER2 shedding. Preferably, the MMP antagonist is a membrane-tethered MMP (MT-MMP) antagonist, such as MT1-MMP (MMP-14), MT2-MMP (MMP-15), MT3-MMP (MMP-16), MT5-MMP (MMP-24), MT4-MMP (MMP-17), or MT6-MMP (MMP-25). Most preferably, the MT-MMP is MMP-15, and desirably, the antagonist binds selectively or preferentially to MMP-15, without significantly binding other proteases or MMPs other than MMP-15, and/or the antagonist interferes with MMP-15 proteolytic function without significantly interfering with function of other proteases or MMPs other than MMP-15.

In the preferred embodiment, the treated cell displays HER and/or MMP expression, amplification, or activation. For example, the cell may display HER2 and/or MMP-15 overexpression or amplification.

The activity of the MMP antagonist may be enhanced by combining it with another anti-tumor drug, HER inhibitor, or HER2 antibody (such as trastuzumab or pertuzumab). Examples of HER inhibitors that may be combined with the MMP antagonist include trastuzumab, pertuzumab, cetuximab, ABX-EGF, EMD7200, gefitinib, erlotinib, CP724714, C11033, GW572016, IMC-11F8, and TAK165.

The invention also concerns a method for reducing HER2 extracellular domain (ECD) serum level in a mammal, comprising administering a matrix met alloprotease (MMP) antagonist to the mammal in an amount effective to reduce the HER2 ECD serum level in the mammal. The mammal optionally has an elevated MMP level.

The invention also provides a method for treating cancer in a mammal comprising administering a matrix met alloprotease (MMP) antagonist to the mammal in an amount effective to treat the cancer. The cancer may display HER and/or MMP expression, amplification, or activation. For example, the cancer may display HER2 or MMP-15 overexpression or amplification. In one embodiment, the treated mammal has an elevated shed HER2 serum level or elevated p95 HER2 level.

This invention also relates to a method for treating a HER inhibitor-resistant cancer in a mammal comprising administering to the mammal a matrix met alloprotease (MMP) antagonist in an amount effective to treat the cancer. For example, the mammal may be resistant to a HER2 antibody, such as trastuzumab.

Also provided is a method for reducing p95 HER2 level in a cell comprising exposing the cell to a matrix met alloprotease (MMP) antagonist in an amount effective to reduce the p95 HER2 level.

Various MMP antagonists may be used, but preferably the MMP antagonist is a small molecule inhibitor or an antibody. Methods for making antibodies are described hereinbelow.

III. Production of Antibodies

A description follows as to exemplary techniques for the production of antibodies used in accordance with the present invention. The antigen to be used for production of antibodies may be, e.g., a soluble form of the antigen or a portion thereof, containing the desired epitope. Alternatively, cells expressing the antigen at their cell surface (e.g. NIH-3T3 cells transformed to overexpress HER2; or a carcinoma cell line such as SK-BR-3 cells, see Stancovski et al. PNAS (USA) 88:8691-8695 (1991)) can be used to generate antibodies. Other forms of antigen useful for generating antibodies will be apparent to those skilled in the art.

(i) Polyclonal Antibodies

Polyclonal antibodies are preferably raised in animals by multiple subcutaneous (sc) or intraperitoneal (ip) injections of the relevant antigen and an adjuvant. It may be useful to conjugate the relevant antigen to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOCl₂, or R¹N═C═NR, where R and R¹ are different alkyl groups.

Animals are immunized against the antigen, immunogenic conjugates, or derivatives by combining, e.g., 100 μg or 5 μg of the protein or conjugate (for rabbits or mice, respectively) with 3 volumes of Freund's complete adjuvant and injecting the solution intradermally at multiple sites. One month later the animals are boosted with ⅕ to 1/10 the original amount of peptide or conjugate in Freund's complete adjuvant by subcutaneous injection at multiple sites. Seven to 14 days later the animals are bled and the serum is assayed for antibody titer. Animals are boosted until the titer plateaus. Preferably, the animal is boosted with the conjugate of the same antigen, but conjugated to a different protein and/or through a different cross-linking reagent. Conjugates also can be made in recombinant cell culture as protein fusions. Also, aggregating agents such as alum are suitably used to enhance the immune response.

(ii) Monoclonal Antibodies

Various methods for making monoclonal antibodies herein are available in the art. For example, the monoclonal antibodies may be made using the hybridoma method first described by Kohler et al., Nature, 256:495 (1975), by recombinant DNA methods (U.S. Pat. No. 4,816,567).

In the hybridoma method, a mouse or other appropriate host animal, such as a hamster, is immunized as hereinabove described to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)).

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high-level production of antibody by the selected antibody-producing cells, and are sensitive to a medium such as HAT medium. Among these, preferred myeloma cell lines are murine myeloma lines, such as those derived from MOPC-21 and MPC-11 mouse tumors available from the Salk Institute Cell Distribution Center, San Diego, Calif. USA, and SP-2 or X63-Ag8-653 cells available from the American Type Culture Collection, Rockville, Md. USA. Human myeloma and mouse-human heteromyeloma cell lines also have been described for the production of human monoclonal antibodies (Kozbor, J. Immunol., 133:3001 (1984); and Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunoabsorbent assay (ELISA).

The binding affinity of the monoclonal antibody can, for example, be determined by the Scatchard analysis of Munson et al., Anal. Biochem., 107:220 (1980).

After hybridoma cells are identified that produce antibodies of the desired specificity, affinity, and/or activity, the clones may be subcloned by limiting dilution procedures and grown by standard methods (Goding, Monoclonal Antibodies: Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal.

The monoclonal antibodies secreted by the subclones are suitably separated from the culture medium, ascites fluid, or serum by conventional antibody purification procedures such as, for example, protein A-Sepharose, hydroxylapatite chromatography, gel electrophoresis, dialysis, or affinity chromatography.

DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as E. coli cells, simian COS cells, Chinese Hamster Ovary (CHO) cells, or myeloma cells that do not otherwise produce antibody protein, to obtain the synthesis of monoclonal antibodies in the recombinant host cells. Review articles on recombinant expression in bacteria of DNA encoding the antibody include Skerra et al., Curr. Opinion in Immunol., 5:256-262 (1993) and Plolckthun, Immunol. Revs., 130:151-188 (1992).

In a flurther embodiment, monoclonal antibodies or antibody fragments can be isolated from antibody phage libraries generated using the techniques described in McCafferty et al, Nature, 348:552-554 (1990). Clackson et al, Nature, 352:624-628 (1991) and Marks et al, J. Mol. Biol., 222:581-597 (1991) describe the isolation of murine and human antibodies, respectively, using phage libraries. Subsequent publications describe the production of high affinity (nM range) human antibodies by chain shuffling (Marks et al., Bio/Technology, 10:779-783 (1992)), as well as combinatorial infection and in vivo recombination as a strategy for constructing very large phage libraries (Waterhouse et al, Nuc. Acids. Res., 21:2265-2266 (1993)). Thus, these techniques are viable alternatives to traditional monoclonal antibody hybridoma techniques for isolation of monoclonal antibodies.

The DNA also may be modified, for example, by substituting the coding sequence for human heavy chain and light chain constant domains in place of the homologous murine sequences (U.S. Pat. No. 4,816,567; and Morrison, et al, Proc. Natl Acad Sci. USA, 81:6851 (1984)), or by covalently joining to the immunoglobulin coding sequence all or part of the coding sequence for a non-immunoglobulin polypeptide.

Typically such non-immunoglobulin polypeptides are substituted for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

(iii) Humanized Antibodies

Methods for humanizing non-human antibodies have been described in the art. Preferably, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al, Science, 239:1534-1536 (1988)), by substituting hypervariable region sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some hypervariable region residues and possibly some FR residues are substituted by residues from analogous sites in rodent antibodies.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence which is closest to that of the rodent is then accepted as the human framework region (FR) for the humanized antibody (Sims et al., J. Immunol, 151:2296 (1993); Chothia et al, J. Mol Biol, 196:901 (1987)). Another method uses a particular framework region derived from the consensus sequence of all human antibodies of a particular subgroup of light or heavy chains. The same framework may be used for several different humanized antibodies (Carter et al., Proc. Natl. Acad. Sci. USA, 89:4285 (1992); Presta et al., J. Immunol., 151:2623 (1993)).

It is further important that antibodies be humanized with retention of high affinity for the antigen and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

(iv) Human Antibodies

As an alternative to humanization, human antibodies can be generated. For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (J_{H}) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Natl. Acad Sci. USA, 90:2551 (1993); Jakobovits et al, Nature, 362:255-258 (1993); Bruggermann et al, Year in Immuno., 7:33 (1993); and U.S. Pat. Nos. 5,591,669, 5,589,369 and 5,545,807.

Alternatively, phage display technology (McCafferty et al., Nature 348:552-553 (1990)) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B-cell. Phage display can be performed in a variety of formats; for their review see, e.g., Johnson, Kevin S. and Chiswell, David J., Current Opinion in Structural Biology 3:564-571 (1993). Several sources of V-gene segments can be used for phage display. Clackson et al., Nature, 352:624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al, J. Mol. Biol. 222:581-597 (1991), or Griffith et al, EMBO J. 12:725-734 (1993). See, also, U.S. Pat. Nos. 5,565,332 and 5,573,905.

As discussed above, human antibodies may also be generated by in vitro activated B cells (see U.S. Pat. Nos. 5,567,610 and 5,229,275).

Human HER2 antibodies are described in U.S. Pat. No. 5,772,997 issued Jun. 30, 1998 and WO. 97/00271 published Jan. 3, 1997.

(v) Antibody Fragments

Various techniques have been developed for the production of antibody fragments comprising one or more antigen binding regions. Traditionally, these fragments were derived via proteolytic digestion of intact antibodies (see, e.g., Morimoto et al., Journal of Biochemical and Biophysical Methods 24:107-117 (1992); and Brennan et al., Science, 229:81 (1985)). However, these fragments can now be produced directly by recombinant host cells. For example, the antibody fragments can be isolated from the antibody phage libraries discussed above. Alternatively, Fab′-SH fragments can be directly recovered from E. coli and chemically coupled to form F(ab′)₂ fragments (Carter et al., Bio/Technology 10: 163-167 (1992)). According to another approach, F(ab′)₂ fragments can be isolated directly from recombinant host cell culture. Other techniques for the production of antibody fragments will be apparent to the skilled practitioner. In other embodiments, the antibody of choice is a single chain Fv fragment (scFv). See WO 93/16185; U.S. Pat. No. 5,571,894; and U.S. Pat. No. 5,587,458. The antibody fragment may also be a “linear antibody”, e.g., as described in U.S. Pat. No. 5,641,870 for example. Such linear antibody fragments may be monospecific or bispecific.

(vi) Bispecific Antibodies

Bispecific antibodies are antibodies that have binding specificities for at least two different epitopes. Exemplary bispecific antibodies may bind to two different epitopes of the HER2 protein. Other such antibodies may combine a HER2 binding site with binding site(s) for EGFR, HER3 and/or HER4. Alternatively, a HER2 arm may be combined with an arm which binds to a triggering molecule on a leukocyte such as a T-cell receptor molecule (e.g. CD2 or CD3), or Fc receptors for IgG (FcγR), such as FcγRI (CD64), FcγRII (CD32) and FcγRIII (CD16) so as to focus cellular defense mechanisms to the HER2-expressing cell. Bispecific antibodies may also be used to localize cytotoxic agents to cells which express HER2. These antibodies possess a HER2-binding arm and an arm which binds the cytotoxic agent (e.g. saporin, anti-interferon-α, vinca alkaloid, ricin A chain, methotrexate or radioactive isotope hapten). Bispecific antibodies can be prepared as full length antibodies or antibody fragments (e.g. F(ab′)₂bispecific antibodies).

WO 96/16673 describes a bispecific HER2/FcγRIII antibody and U.S. Pat. No. 5,837,234 discloses a bispecific HER2/FcγRI antibody IDMI (Osidem). A bispecific HER2/Fcα antibody is shown in WO98/02463. U.S. Pat. No.5,821,337 teaches a bispecific HER2/CD3 antibody. MDX-210 is a bispecific HER2-FcγRIII Ab.

Methods for making bispecific antibodies are known in the art. Traditional production of full length bispecific antibodies is based on the coexpression of two immunoglobulin heavy chain-light chain pairs, where the two chains have different specificities (Milistein et al., Nature, 305:537-539 (1983)). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829, and in Traunecker et al., EMBO J., 10:3655-3659 (1991).

According to a different approach, antibody variable domains with the desired binding specificities (antibody-antigen combining sites) are fuised to immunoglobulin constant domain sequences. The fusion preferably is with an immunoglobulin heavy chain constant domain, comprising at least part of the hinge, CH2, and CH3 regions. It is preferred to have the first heavy-chain constant region (CH1) containing the site necessary for light chain binding, present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In a preferred embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690. For further details of generating bispecific antibodies see, for example, Suresh et al., Methods in Enzymology, 121:210 (1986).

According to another approach described in U.S. Pat. No. 5,731,168, the interface between a pair of antibody molecules can be engineered to maximize the percentage of heterodimers which are recovered from recombinant cell culture. The preferred interface comprises at least a part of the C_{H}3 domain of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373, and EP 03089). Heteroconjugate antibodies may be made using any convenient cross-linking methods. Suitable cross-linking agents are well known in the art, and are disclosed in U.S. Pat. No. 4,676,980, along with a number of cross-linking techniques.

Techniques for generating bispecific antibodies from antibody fragments have also been described in the literature. For example, bispecific antibodies can be prepared using chemical linkage. Brennan et al., Science, 229: 81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Recent progress has facilitated the direct recovery of Fab′-SH fragments from E. coli, which can be chemically coupled to form bispecific antibodies. Shalaby et al., J. Exp. Med, 175: 217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the HER2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol., 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad Sci. USA, 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See Gruber et al., J. Immunol., 152:5368 (1994).

Antibodies with more than two valencies are contemplated. For example, trispecific antibodies can be prepared. Tutt et al. J. Immunol. 147: 60 (1991).

(vii) Other Amino Acid Sequence Modifications

Amino acid sequence modification(s) of the antibodies described herein are contemplated. For example, it may be desirable to improve the binding affinity and/or other biological properties of the antibody. Amino acid sequence variants of the antibody are prepared by introducing appropriate nucleotide changes into the antibody nucleic acid, or by peptide synthesis. Such modifications include, for example, deletions from, and/or insertions into and/or substitutions of, residues within the amino acid sequences of the antibody. Any combination of deletion, insertion, and substitution is made to arrive at the final construct, provided that the final construct possesses the desired characteristics. The amino acid changes also may alter post-translational processes of the antibody, such as changing the number or position of glycosylation sites.

A useful method for identification of certain residues or regions of the antibody that are preferred locations for mutagenesis is called “alanine scanning mutagenesis” as described by Cunningham and Wells Science, 244:1081-1085 (1989). Here, a residue or group of target residues are identified (e.g., charged residues such as arg, asp, his, lys, and glu) and replaced by a neutral or negatively charged amino acid (most preferably alanine or polyalanine) to affect the interaction of the amino acids with antigen. Those amino acid locations demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, ala scanning or random mutagenesis is conducted at the target codon or region and the expressed antibody variants are screened for the desired activity.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include antibody with an N-terminal methionyl residue or the antibody fused to a cytotoxic polypeptide. Other insertional variants of the antibody molecule include the fusion to the N- or C-terminus of the antibody to an enzyme (e.g. for ADEPT) or a polypeptide which increases the serum half-life of the antibody.

Another type of variant is an amino acid substitution variant. These variants have at least one amino acid residue in the antibody molecule replaced by a different residue. The sites of greatest interest for substitutional mutagenesis include the hypervariable regions, but FR alterations are also contemplated.

Any cysteine residue not involved in maintaining the proper conformation of the antibody also may be substituted, generally with serine, to improve the oxidative stability of the molecule and prevent aberrant crosslinking. Conversely, cysteine bond(s) may be added to the antibody to improve its stability (particularly where the antibody is an antibody fragment such as an Fv fragment).

A particularly preferred type of substitutional variant involves substituting one or more hypervariable region residues of a parent antibody (e.g. a humanized or human antibody). Generally, the resulting variant(s) selected for further development will have improved biological properties relative to the parent antibody from which they are generated. A convenient way for generating such substitutional variants involves affinity maturation using phage display. Briefly, several hypervariable region sites (e.g. 6-7 sites) are mutated to generate all possible amino substitutions at each site. The antibody variants thus generated are displayed in a monovalent fashion from filamentous phage particles as fusions to the gene III product of M13 packaged within each particle. The phage-displayed variants are then screened for their biological activity (e.g. binding affinity) as herein disclosed. In order to identify candidate hypervariable region sites for modification, alanine scanning mutagenesis can be performed to identify hypervariable region residues contributing significantly to antigen binding. Alternatively, or additionally, it may be beneficial to analyze a crystal structure of the antigen-antibody complex to identify contact points between the antibody and human HER2. Such contact residues and neighboring residues are candidates for substitution according to the techniques elaborated herein. Once such variants are generated, the panel of variants is subjected to screening as described herein and antibodies with superior properties in one or more relevant assays may be selected for further development.

Another type of amino acid variant of the antibody alters the original glycosylation pattern of the antibody. By altering is meant deleting one or more carbohydrate moieties found in the antibody, and/or adding one or more glycosylation sites that are not present in the antibody.

Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Addition of glycosylation sites to the antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

Where the antibody comprises an Fc region, the carbohydrate attached thereto may be altered. For example, antibodies with a mature carbohydrate structure that lacks fticose attached to an Fc region of the antibody are described in US Pat Appl No US 2003/0157108 A1, Presta, L. See also US 2004/0093621 A1 (Kyowa Hakko Kogyo Co., Ltd). Antibodies with a bisecting N-acetylglucosamine (GlcNAc) in the carbohydrate attached to an Fc region of the antibody are referenced in WO03/011878, Jean-Mairet et al. and U.S. Pat. No. 6,602,684, Umana et al. Antibodies with at least one galactose residue in the oligosaccharide attached to an Fc region of the antibody are reported in WO97/30087, Patel et al. See, also, WO98/58964 (Raju, S.) and WO99/22764 (Raju, S.) concerning antibodies with altered carbohydrate attached to the Fc region thereof.

It may be desirable to modify the antibody of the invention with respect to effector function, e.g. so as to enhance antigen-dependent cell-mediated cyotoxicity (ADCC) and/or complement dependent cytotoxicity (CDC) of the antibody. This may be achieved by introducing one or more amino acid substitutions in an Fc region of the antibody. Alternatively or additionally, cysteine residue(s) may be introduced in the Fc region, thereby allowing interchain disulfide bond formation in this region. The homodimeric antibody thus generated may have improved internalization capability and/or increased complement-mediated cell killing and antibody-dependent cellular cytotoxicity (ADCC). See Caron et al., J. Exp Med 176:1191-1195 (1992) and Shopes, B. J. Immunol. 148:2918-2922 (1992). Homodimeric antibodies with enhanced anti-tumor activity may also be prepared using heterobifunctional cross-linkers as described in Wolff et al. Cancer Research 53:2560-2565 (1993). Alternatively, an antibody can be engineered which has dual Fc regions and may thereby have enhanced complement lysis and ADCC capabilities. See Stevenson et al. Anti-Cancer Drug Design 3:219-230 (1989).

WO00/42072 (Presta, L.) describes antibodies with improved ADCC function in the presence of human effector cells, where the antibodies comprise amino acid substitutions in the Fc region thereof. Preferably, the antibody with improved ADCC comprises substitutions at positions 298, 333, and/or 334 of the Fc region. Preferably the altered Fc region is a human IgG1 Fc region comprising or consisting of substitutions at one, two or three of these positions.

Antibodies with altered Cl q binding and/or complement dependent cytotoxicity (CDC) are described in WO99/51642, U.S. Pat. No. 6,194,551B1, U.S. Pat. No. 6,242,195B1, U.S. Pat. No. 6,528,624B1 and U.S. Pat. No. 6,538,124 (Idusogie et al.). The antibodies comprise an amino acid substitution at one or more of amino acid positions 270, 322, 326, 327, 329, 313, 333 and/or 334 of the Fc region thereof (using Eu numbering of Fc region residues as in Kabat).

To increase the serum half life of the antibody, one may incorporate a salvage receptor binding epitope into the antibody (especially an antibody fragment) as described in U.S. Pat. No. 5,739,277, for example. As used herein, the term “salvage receptor binding epitope” refers to an epitope of the Fc region of an IgG molecule (e.g., IgG₁, IgG₂, IgG₃, or IgG₄) that is responsible for increasing the in vivo serum half-life of the IgG molecule.

Antibodies with improved binding to the neonatal Fc receptor (FcRn), and increased half-lives, are described in WO00/42072 (Presta, L.) and US2005/0014934A1 (Hinton et al.). These antibodies comprise an Fc region with one or more substitutions therein which improve binding of the Fc region to FcRn. For example, the Fc region may have substitutions at one or more of positions 238, 250, 256, 265, 272, 286, 303, 305, 307, 311, 312, 314,317, 340, 356, 360, 362, 376, 378, 380, 382, 413, 424, 428 or 434 thereof (using Eu numbering of Fc region residues as in Kabat). The preferred Fc region-comprising antibody variant with improved FcRn binding comprises amino acid substitutions at one, two or three of positions 307, 380 and 434 of the Fc region thereof.

Engineered antibodies with three or more (preferably four) functional antigen binding sites are also contemplated (US Appln No. US2002/0004587 A1, Miller et al.).

Nucleic acid molecules encoding amino acid sequence variants of the antibody are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the antibody.

(viii) Immunoconjugates

The invention also pertains to immunoconjugates comprising an antibody conjugated to a cytotoxic agent such as a chemotherapeutic agent, toxin (e.g. a small molecule toxin or an enzymatically active toxin of bacterial, fungal, plant or animal origin, including fragments and/or variants thereof), or a radioactive isotope (i.e., a radioconjugate).

Chemotherapeutic agents useful in the generation of such immunoconjugates have been described above. Conjugates of an antibody and one or more small molecule toxins, such as a calicheamicin, a maytansine (U.S. Pat. No. 5,208,020), a trichothene, and CC 1065 are also contemplated herein.

In one preferred embodiment of the invention, the antibody is conjugated to one or more maytansine molecules (e.g. about 1 to about 10 maytansine molecules per antibody molecule). Maytansine may, for example, be converted to May-SS-Me which may be reduced to May-SH3 and reacted with modified antibody (Chari et al. Cancer Research 52: 127-131 (1992)) to generate a maytansinoid-antibody immunoconjugate.

Another immunoconjugate of interest comprises an antibody conjugated to one or more calicheamicin molecules. The calicheamicin family of antibiotics are capable of producing double-stranded DNA breaks at sub-picomolar concentrations. Structural analogues of calicheamicin which may be used include, but are not limited to, γ₁^{I}, α₂^{I}, α₃^{I}, N-acetyl-γ_{I}¹, PSAG and θ^{I}₁ (Hinman et al. Cancer Research 53: 3336-3342 (1993) and Lode et al. Cancer Research 58: 2925-2928 (1998)). See, also, U.S. Pat. Nos. 5,714,586; 5,712,374; 5,264,586; and 5,773,001 expressly incorporated herein by reference.

Enzymatically active toxins and fragments thereof which can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin and the tricothecenes. See, for example, WO 93/21232 published Oct. 28, 1993.

The present invention further contemplates an immunoconjugate formed between an antibody and a compound with nucleolytic activity (e.g. a ribonuclease or a DNA endonuclease such as a deoxyribonuclease; DNase).

A variety of radioactive isotopes are available for the production of radioconjugated HER2 antibodies. Examples include At²¹¹, I¹³¹, I¹²⁵, Y⁹⁰, Re¹⁸⁶, Re¹⁸⁸, S,¹⁵³, Bi²¹², P³² and radioactive isotopes ofLu.

Conjugates of the antibody and cytotoxic agent may be made using a variety of bifunctional protein coupling agents such as N-succinimidyl-3-(2-pyridyldithiol) propionate (SPDP), succinimidyl4-(N-maleimidomethyl) cyclohexane-1-carboxylate, iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al. Science 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026. The linker may be a “cleavable linker” facilitating release of the cytotoxic drug in the cell. For example, an acid-labile linker, peptidase-sensitive linker, dimethyl linker or disulfide-containing linker (Chari et al. Cancer Research 52: 127-131 (1992)) may be used.

Alternatively, a fusion protein comprising the antibody and cytotoxic agent may be made, e.g. by recombinant techniques or peptide synthesis.

Other immunoconjugates are contemplated herein. For example, the antibody may be linked to one of a variety of nonproteinaceous polymers, e.g., polyethylene glycol, polypropylene glycol, polyoxyalkylenes, or copolymers of polyethylene glycol and polypropylene glycol. The antibody also may be entrapped in microcapsules prepared, for example, by coacervation techniques or by interfacial polymerization (for example, hydroxymethylcellulose or gelatin-microcapsules and poly-(methylmethacylate) microcapsules, respectively), in colloidal drug delivery systems (for example, liposomes, albumin microspheres, microemulsions, nano-particles and nanocapsules), or in macroemulsions. Such techniques are disclosed in Remington's Pharmaceutical Sciences, 16th edition, Oslo, A., Ed., (1980).

The antibodies disclosed herein may also be formulated as immunoliposomes. Liposomes containing the antibody are prepared by methods known in the art, such as described in Epstein et al., Proc. Natl. Acad Sci. USA, 82:3688 (1985); Hwang et al., Proc. Natl Acad Sci. USA, 77:4030 (1980); U.S. Pat. Nos. 4,485,045 and 4,544,545; and WO97/38731 published Oct. 23, 1997. Liposomes with enhanced circulation time are disclosed in U.S. Pat. No. 5,013,556.

Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al. J. Biol. Chem. 257: 286-288 (1982) via a disulfide interchange reaction. A chemotherapeutic agent is optionally contained within the liposome. See Gabizon et al. J. National Cancer Inst. 81(19)1484 (1989).

IV. Pharmaceutical Formulations

Therapeutic formulations of the MMP antagonists used in accordance with the present invention are prepared for storage by mixing a MMP antagonist having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; met al complexes (e.g. Zn-protein complexes); and/or non-ionic surfactants such as TWEEN™, PLURONICS™ or polyethylene glycol (PEG). Lyophilized antibody formulations are described in WO 97/04801, expressly incorporated herein by reference.

The formulation herein may also contain more than one active compound as necessary for the particular indication being treated, preferably those with complementary activities that do not adversely affect each other. Various drugs which can be combined with the MMP antagonist are described in the method of treatment section below. Such molecules are suitably present in combination in amounts that are effective for the purpose intended.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

The formulations to be used for in vivo administration must be sterile. This is readily accomplished by filtration through sterile filtration membranes.

V. Treatment

Examples of various cancers that can be treated with the MMP antagonist are listed in the definition section above. Administration of the MMP antagonist will result in an improvement in the signs or symptoms of cancer.

The MMP antagonist is administered to a human patient in accord with known methods, such as intravenous administration, e.g., as a bolus or by continuous infusion over a period of time, by intramuscular, intraperitoneal, intracerobrospinal, subcutaneous, intra-articular, intrasynovial, intrathecal, oral, topical, or inhalation routes.

For the prevention or treatment of disease, the dose of MMP antagonist will depend on the type of cancer to be treated, as defined above, the severity and course of the cancer, whether the antibody is administered for preventive or therapeutic purposes, previous therapy, the patient's clinical history and response to the antibody, and the discretion of the attending physician.

While the MMP antagonist may be the sole anti-tumor drug administered, the patient is optionally treated with a combination of the MMP antagonist, and one or more other anti-tumor agent(s). The combined administration includes coadministration or concurrent administration, using separate formulations or a single pharmaceutical formulation, and consecutive administration in either order, wherein preferably there is a time period while both (or all) active agents simultaneously exert their biological activities. Thus, the other anti-tumor agent may be administered prior to, or following, administration of the MMP antagonist. In this embodiment, the timing between at least one administration of the MMP antagonist and at least one administration of the other anti-tumor agent is preferably approximately 1 month or less, and most preferably approximately 2 weeks or less. Alternatively, the MMP antagonist and other anti-tumor agent are administered concurrently to the patient, in a single formulation or separate formulations. Treatment with the combination of the MMP antagonist and the other anti-tumor agent may result in a synergistic, or greater than additive, therapeutic benefit to the patient.

Examples of second anti-tumor agents that may be combined with the MMP antagonist include: one or more chemotherapeutic agent(s); a HER inhibitor (e.g trastuzumab, pertuzumab, cetuximab, ABX-EGF, EMD7200, gefitinib, erlotinib, CP724714, CI1033, GW572016, IMC-11F8, TAK165, etc); Raf and/or ras inhibitor (see, for example, WO 2003/86467); a growth inhibitory HER2 antibody such as trastuzumab; a HER dimerization inhibitor such as Pertuzumab; a HER2 antibody which induces apoptosis of a HER2-overexpressing cell, such as 7C2, 7F3 or humanized variants thereof; an antibody directed against a tumor associated antigen, such as EGFR, HER3, HER4; anti-hormonal compound, e.g., an anti-estrogen compound such as tamoxifen, or an aromatase inhibitor; a cardioprotectant (to prevent or reduce any myocardial dysfunction associated with the therapy); a cytokine; an EGFR-targeted drug (such as TARCEVA®, IRESSA® or Cetuximab); an anti-angiogenic agent (especially bevacizumab sold by Genentech under the trademark AVASTIN™); a tyrosine kinase inhibitor; a COX inhibitor (for instance a COX-1 or COX-2 inhibitor); non-steroidal anti-inflammatory drug, Celecoxib (CELEBREX®); farnesyl transferase inhibitor (for example, Tipifarnib/ZARNESTRA® R115777 available from Johnson and Johnson or Lonafamib SCH66336 available from Schering-Plough); antibody that binds oncofet al protein CA 125 such as Oregovomab (MoAb B43.13); HER2 vaccine (such as HER2 AutoVac vaccine from Pharmexia, or APC8024 protein vaccine from Dendreon, or HER2 peptide vaccine from GSK/Corixa); doxorubicin HCl liposome injection (DOXIL®); topoisomerase I inhibitor such as topotecan; taxane; HER2 and EGFR dual tyrosine kinase inhibitor such as lapatinib/GW572016; TLK286 (TELCYTA®); EMD-7200; a temperature-reducing medicament such as acetaminophen, diphenhydramine, or meperidine; hematopoietic growth factor, etc.

Suitable dosages for any of the above coadministered agents are those presently used and may be lowered due to the combined action (synergy) of the agent and MMP antagonist.

In addition to the above therapeutic regimes, the patient may be subjected to surgical removal of cancer cells and/or radiation therapy.

Aside from administration of protein MMP antagonists to the patient, the present application contemplates administration of MMP antagonists by gene therapy. See, for example, WO96/07321 published Mar. 14, 1996 concerning the use of gene therapy to generate intracellular antibodies.

There are two major approaches to getting the nucleic acid (optionally contained in a vector) into the patient's cells; in vivo and ex vivo. For in vivo delivery the nucleic acid is injected directly into the patient, usually at the site where the antibody is required. For ex vivo treatment, the patient's cells are removed, the nucleic acid is introduced into these isolated cells and the modified cells are administered to the patient either directly or, for example, encapsulated within porous membranes which are implanted into the patient (see, e.g. U.S. Pat. Nos. 4,892,538 and 5,283,187). There are a variety of techniques available for introducing nucleic acids into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. A commonly used vector for ex vivo delivery of the gene is a retrovirus.

The currently preferred in vivo nucleic acid transfer techniques include transfection with viral vectors (such as adenovirus, Herpes simplex I virus, or adeno-associated virus) and lipid-based systems (useful lipids for lipid-mediated transfer of the gene are DOTMA, DOPE and DC-Chol, for example). In some situations it is desirable to provide the nucleic acid source with an agent that targets the target cells, such as an antibody specific for a cell surface membrane protein or the target cell, a ligand for a receptor on the target cell, etc. Where liposomes are employed, proteins which bind to a cell surface membrane protein associated with endocytosis may be used for targeting and/or to facilitate uptake, e.g. capsid proteins or fragments thereof tropic for a particular cell type, antibodies for proteins which undergo internalization in cycling, and proteins that target intracellular localization and enhance intracellular half-life. The technique of receptor-mediated endocytosis is described, for example, by Wu et al., J. Biol. Chem. 262:44294432 (1987); and Wagner et al., Proc. Natl. Acad Sci USA 87:3410-3414 (1990). For review of the currently known gene marking and gene therapy protocols see Anderson et al., Science 256:808-813 (1992). See also WO 93/25673 and the references cited therein.

VI. Deposit of Materials

The following hybridoma cell lines have been deposited with the American Type Culture Collection, 10801 University Boulevard, Manassas, Va. 20110-2209, USA (ATCC):

| Antibody Designation | ATCC No. | Deposit Date |
|---|---|---|
| 7C2 | ATCC HB-12215 | Oct. 17, 1996 |
| 7F3 | ATCC HB-12216 | Oct. 17, 1996 |
| 4D5 | ATCC CRL 10463 | May 24, 1990 |
| 2C4 | ATCC HB-12697 | Apr. 8, 1999 |

Further details of the invention are illustrated by the following non-limiting Examples. The disclosures of all citations in the specification are expressly incorporated herein by reference.

### EXAMPLE 1

The following example investigated the role of matrix met alloproteases (MMPs) in HER2 shedding.

Materials and Methods

Cell Culture and Transfections

All cell lines used in this study were obtained from the American Type Tissue Culture Collection (ATCC, Manassas, Va.). BT 474, MCF-7, and SKBR-3 cells were maintained in high-glucose DMEM:Ham's F12 (50:50) supplemented with 10% heat-inactivated FBS and 2 mM L-Glutamine. Cos-7 cells were maintained in High-Glucose DMEM supplemented with 10% heat-inactivated FBS and 2 mM L-Glutamine. Cell lines were maintained at 37° C. in a humidified incubator supplied with 5% CO₂. BT 474 and SKBR-3 cells were transiently transfected with constructs as described by electroporation using kit V according to the manufacturer's instructions (AMAXA™). Cos-7 cells were transfected as described using LIPOFECTAMINE™ 2000 (Invitrogen) according to the manufacturer's recommendations.

Fo5 and f2:1282 Mouse Xenograft Tumor lines

The Fo5 and f2: 1282 lines have been previously described (Finkle et al., Clin. Cancer Res. 10: 2499-2511 (2004)). These lines were derived from primary tumors from a MMTV HER2 transgenic mouse and passaged in the mammary fat pad of FVB mice.

GM6001 Injections

Fo5 tumors were transplanted into FVB mice and allowed to grow to an average size of 400-600 mm³ prior to initiating the study. GM6001 (3-[N-hydroxycarbomyl]-[2R]-isobutylpropionyl-L-tryptophan methylamide) was obtained from Calbiochem and reconstituted in a slurry of 4% carboxymethylcellulose/0.9% PBS and administered daily by intraperitoneal injection at 100 mg/kg body weight for 3 days. Twenty-four hours after the third day of injection, animals were sacrificed and serum collected by cardiac puncture. Tumors size was measured at day 0 and at day 4. Tumors were removed and flash frozen for further analysis.

RNA Preparation and AFFYMETRIX™ Arrays

Total RNA was isolated from flash frozen tumor samples using a RNAEASY™ kit (Qiagen, Chatsworth, Calif.). Residual genomic DNA was removed by DNAse I treatment (Roche Molecular Biochemicals). Microarray experiments were performed and analyzed as previously described (Jin, H et al., Circulation 103:736-742 (2001)). Samples were hybridized to the AFFYMETRIX™ mouse genome single array (MOE430P) and known genes array (MOE430A) (AFFYMETRIX™, Inc., Santa Clara, Calif.). Experiments were done in three replicates for three Fo5 and three f2:1282 tumor samples. A Mann-Whitney pairwise comparison was performed and those genes with a 2-fold increase in expression having a concordance of greater than 80% were considered significant. The GeneLogic Bioexpress Database (GeneLogic, Gaithersburg, Md.), a collection of gene expression data from AFFYMETRIX™ microarray analysis of cell lines and tissues, was used to examine the expression of proteases identified from the Fo5-f2:1282 tumor differential screen for expression in SKBR-3 and BT 474 cell lines.

HER2 ECD ELISA

Serun from mice harboring Fo5 or f2: 1282 xenograft tumors was collected by cardiac puncture and HER2 ECD levels detected using a previously described HER2 ELISA (Finkle et al. Clin. Cancer Res. 10: 2499-2511 (2004)). Serum was diluted 1:50 using assay buffer (PBS/0.5% BSA, 0.05% TWEEN® 20/10 ppm PROCLIN® 300/0.2% BGG/0.25% CHAPS/0.15 M NaCl/5mM EDTA (pH 7.4) followed by additional 1:2 serial dilutions. Shed HER2 was captured using a goat anti-HER2 polyclonal antibody (Genentech) onto NUNC® Maxisorp plates and detected using a biotin-conjugated rabbit anti-HER2 polyclonal antibody (Genentech) followed by a AMDEX™-strepavidin-horseradish peroxidase antibody (Amersham Pharmacia Biotech) following a procedure described previously (Sias et al., J. Immunol. Meth. 109:219-27 (1990)). For cell culture experiments, cells were treated as indicated and conditioned media was collected and diluted in assay buffer serially 1:2. The concentration of shed HER2 in serum or in conditioned media was determined from a four-parameter fit of a standard curve using purified recombinant HER2 ECD protein (Genentech) as a standard.

DNA Constructs and Protein Purification

Human MMP-15 was cloned into pRK5 by PCR using an origen clone as a template. A C-terminally VSHis tagged full length version was generated by PCR using primers

5′-GCCGAAGCTTGCCACCATGGGCAGCGACCCGAGCGCG-3′ (SEQ ID NO. 9) and

5′-GCTGTCTAGAGTTCACCGTCCGTGCCAGTGCCACCTCCTCC-3′ (SEQ ID NO. 10)

and cloned in pcDNA3.IV5His.

A soluble version of MMP-15, which lacks the transmembrane domain, was generated by PCR using primers

5′-GCCGAAGCTTGCCACCATGGGCAGCGACCCGAGCGCG-3′ (SEQ ID NO. 11) and

5′-GCTGTCTAGAGACCCACTCCTGCAGCGAGCG-3′ (SEQ ID NO. 12).

A catalytically dead mutant of MMP-15 was generated by substituting an alanine residue for glutamine at amino acid 260 of the mature protein by site-directed mutagenesis.

pRK5.gDHER2-Fc (pRK5.gDHER2-IgG) was generated by PCR using primers

5′-CGAGTCGACCTCGAGGCCAGCCCTCTGACGTCC-3′ (SEQ ID NO. 13) and

5′-GGCACGCGTCGTCAGAGGGCTGGCTCTCTG-3′ (SEQ ID NO. 14) and cloned into the XhoI-Mlu digested pRK5.gDHER2-Fc (Fitzpatrick et. al., FEBBS Lett. 431(1):102-6 (1998)). This introduced the putative cleavage site of HER2 identified from purified HER2 ECD isolated from SKBR-3 conditioned media (Yuan et al., Protein Expression and Purification 29:217-222 (2003)).

A truncated version of gDHER2-Fc was also generated that only contains domain IV of HER2 using PCR primers

5′-GGCCTCGAGGGCCTGGCCTGCCACCAG-3′ (SEQ ID NO. 15) and

5′-GGCACGCGTCGTCAGAGGGCTGGCTCTCTG-3′ (SEQ ID NO. 16).

pRK5.HER3-Fc and pRK5.HER4-Fc have been previously described (Fitzpatrick et al., FEBBS Lett. 431(1): 102-6 (1998)). A FLAG epitope (Asp-Tyr-Lys-Asp-Asp-Asp-Asp-Lys; (SEQ ID NO. 17)) was introduced into the N-terminus of pRK5. HER-Fc fusion proteins were transfected into 293 cells and purified from serum free conditioned media as previously described (Fitzpatrick et. al., FEBBS Lett., 431(1): 102-6 (1998)). Soluble MMP-15V5His and sMMP-15V5His(E260A) protein was generated from transiently transfected 293 cell conditioned medium and purified using Ni-NTA agarose according to manufacturer's recommendations (Qiagen).

shRNA Knockdown of MMP-15

Several different shRNAs were evaluated for the ability to reduce MMP-15 protein levels when introduced into cells. The most effective shRNA directed against human MMP-15 was found to recognize bp 5′-CCACCATCTGACCTTTAGCTT-3′ (SEQ ID NO. 18) corresponding to nt 1396-1414 (GENBANK™ accession number NM_{—}002428). The RNAi to MMP-15 was introduced and into the BamHI-EcoRi sites pSIREN vector (BD Biosciences) as oligos:

5′-GATCCGCCACCATCTGACCTTTAGCTTCAAGAGAGCTAAAGGTCAGATGGTGGTTTTTTGCTAGCG-3′ (SEQ ID NO. 19) and

5′-AATTCGCTAGCAAAAAACCACCATCTGACCTTTAGCTCTCTTGAAGCTAAAGGTCAGATGGTGGCG-3′ (SEQ ID NO. 20) which contain an internal hairpin to form a shRNA. A shRNA to MMP-25 was purchased from OPENBIOSYSTEMS™. The pSIREN shRNA construct(s) or vector alone were transiently transfected into SKBR-3 and BT 474 cells using AMAXA™ kit V following manufacturer's recommendations. After 48 hours, conditioned media was collected and assayed for shed HER2 by HER2 ELISA and the cells were lysed directly on the plate using 2×Sample buffer. MMP-15, full length HER2, and p95 HER2 proteins were detected by Western blotting using a rabbit polyclonal anti-MMP-15 antibody (Labvision cat. no. RB-1546-P) or mouse monoclonal anti-HER2 antibody Ab-15 (Labvision, cat. no. MS-599-PO).

In Vitro HER2 Cleavage Assay

The ability of candidate proteases to cleave HER2 ECD was determined in vitro using purified gDHER2-Fc fusion protein as a substrate. The purified catalytic domains of MTI-MMP (MMP-14), MT2-MMP (MMP-15), MT3-MMP (MMP-16), MT4-MMP (MMP-19), and MT5-MMP (MMP-25) were purchased from R&D systems. MMP purified proteins were mixed with gDHER2-Fc, or other HER-Fc proteins as described, in assay buffer (100 mM Tris (pH=7.4), 100 mM NaCl, 2.5 μM ZnCl₂, 10 mM CaCl₂, 0.001% Brij35) in a ratio of 1:100, enzyme:substrate, and incubated at 37° C. for 20 minutes. Reactions were stopped using an equal volume of 2× Sample buffer (Invitrogen), and boiled for 5 min. prior to loading onto a 4-20% Tris-glycine gradient gel (Invitrogen). Proteins were visualized by staining with GEL CODE BLUE™ stain reagent (Pierce cat. no. 24592) following manufacturer's recommendations. The MMP cleavage site was determined by N-terminal protein sequencing by automated Edman degradation using an automated protein sequencer (Kishiyama, A., Anal. Chem. 72(21):5431-6 (2000)).

Immunoprecipitation Assays and Western Blotting

Cos-7 cells were co-transfected with pRK5.Flag-HER2 and pcDNA3.MMP-15 constructs or pcDNA3.1 vector alone using LIPOFECTAMINE™ 2000. Cells were allowed to recover for 48 hrs. Transfected cells were rinsed with PBS and cells were ruptured in lysis buffer (1%TRITON X-100™, 50 mMTris (pH=7.4), 150 mM NaCl, 1 mM PMSF, 10 μg/ml leupeptin, 10 U/ml aprotinin, and 2 mM Na₂VO₄). Lysates were cleared of insoluble material by centrifugation and total protein levels were determined using a BCA protein assay kit (Pierce cat. no. 23229). Two-hundred micrograms of total cellular protein was added to lysis buffer to a final volume of 1 ml and Flag-HER2 was immunoprecipitated using anti-Flag M2-agarose (Sigma) or with polyclonal anti-MMP-15 antibody complexed to ProteinA/G agarose. Immune complexes were washed twice with lysis buffer and resuspended in SDS sample buffer and boiled. Samples were separated on a 4-12% Tris-glycine gradient gel (Invitrogen) and transferred to nitrocellulose membranes. Blots were blocked in 5%BSA/TBST and probed with antibodies to either MMP-15 or HER2 followed by a peroxidase conjugated anti-mouse or rabbit secondary antibody as described. Blots were developed by enhanced chemiluminescence (ECL, Amersham Pharmacia Biotech).

Fo5 and f2:1282 tumors were resuspended in lysis buffer with protease inhibitors and homogenized using a POLYTRON TISSUEMIZER™ (PT2 100) on ice. Tumor lysates were cleared of insoluble material by centrifugation and total protein levels were determined using a BCA protein assay kit. HER2 was imnmunoprecipitated from 1 mg total protein from at least three independent tumor lysates using mouse monoclonal antibody Ab-15 (Labvision, cat. no. MS-599-PO) complexed to Protein A/G sepharaose overnight at 4° C. Complexes were pelleted by centrifugation, washed twice with lysis buffer and resuspended in SDS sample buffer and boiled. Samples were separated on a 4-12% Tris-Glycine gel and transferred to nitrocellulose membranes. Blots were probed with mouse monoclonal antibody Ab-18 to detect phosphorylated HER2 (Labvision, cat. no. MS-1072-P) or with Ab-15 (Labvsion cat. no., MS-599-PO). Expression of MMP-14, MMP-15, and MMP-25 was evaluated by Western blotting from 50 μg of tumor lysate or from 50 ug from BT 474, or MCF-7, or SKBR-3 cellular lysate using rabbit polyclonal Ab-1 (Labvision cat. no. RB-1544-P) to detect MMP-14, rabbit polyclonal Ab-1 (Labvision cat. no. RB-1546-P) to detect MMP-15,or rabbit polyclonal Ab-l (Oncogene Research Products cat. no. PC499) to detect MMP-25.

Activated MAPK (Cell Signaling Technology cat. no. 9101), PhosphoAkt Ser473 (Cell Signaling Technology cat. no. 9271), total Akt (Cell Signaling Technology cat. no. 9272), total Erk (Cell Signaling Technology cat. no. 9102, or phosphoHER3 (Cell Signaling Technology cat. no. 4791) was determined by Western blotting of 50 μg of cellular lysate.

Proliferation Assays

Cells were seeded into 96-well dishes (Nunc) at a density of 10⁴ cells per well in quadruplicate and allowed to adhere overnight. Cells were treated as indicated, or transfected as indicated. Following a 3 day incubation, 25 μl of Alamar blue reagent (Trek Diagnostic Systems, cat. no. 00-100) was added to each well, and incubated for an additional 3 hours. Plates were read in a flourometer with excitation wavelength of 530 nm and emission wavelength of 590 nm according to manufacturer's recommendations. Proliferation relative to non-stimulated or control transfected cells was determined as previously described (Lewis et al., Cancer Res. 56(6):1457-65 (1996)).

Results

The ECD of HER2 is proteolytically shed from breast carcinoma cells in culture and has been detected in the serum of patients with metastatic breast cancer, where it is associated with a poor clinical prognosis (Molina et al. Cancer Res. 61: 4744-4749 (2001)). However, the biological role of HER2 shedding is unclear. The experiments herein were carried out in order to identify the HER2 sheddase.

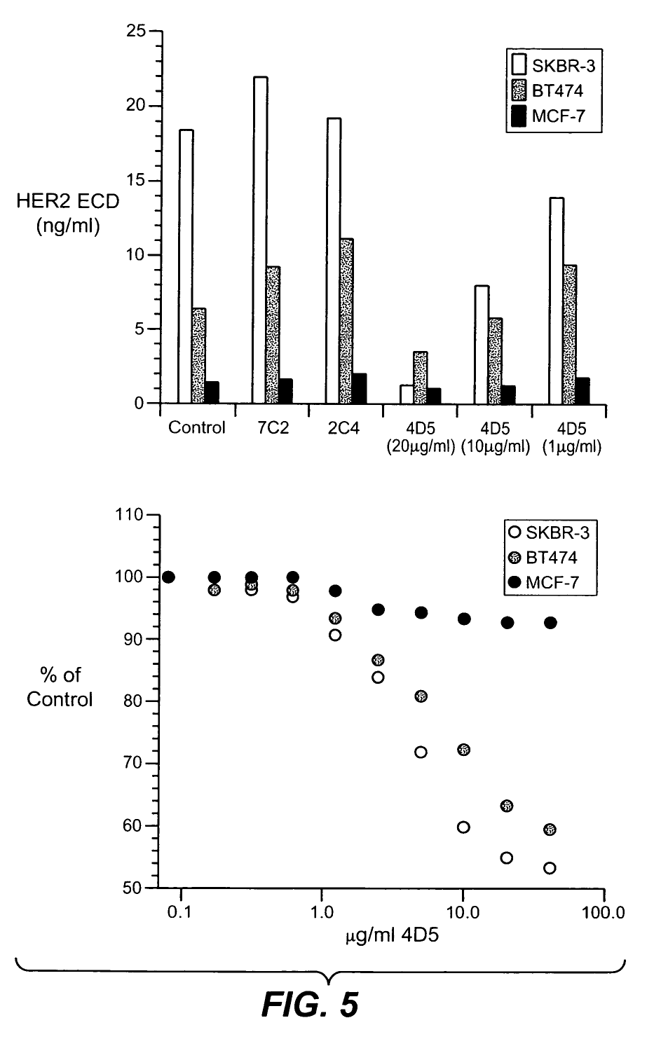

Identification of the HER2 sheddase may also be important as trastuzumab has previously been demonstrated to inhibit shedding in breast carcinoma cell lines (Molina et al. Cancer Res. 61:47444749 (2001)). Consistent with previously reported results, trastuzumab reduced shedding in the breast cancer cell lines BT 474 and SKBR-3 cells by more than 60% at a concentration of 10 μg/ml (FIG. 5, left panel) and reduced cellular proliferation in these cell lines by 50% (FIG. 5, right panel).

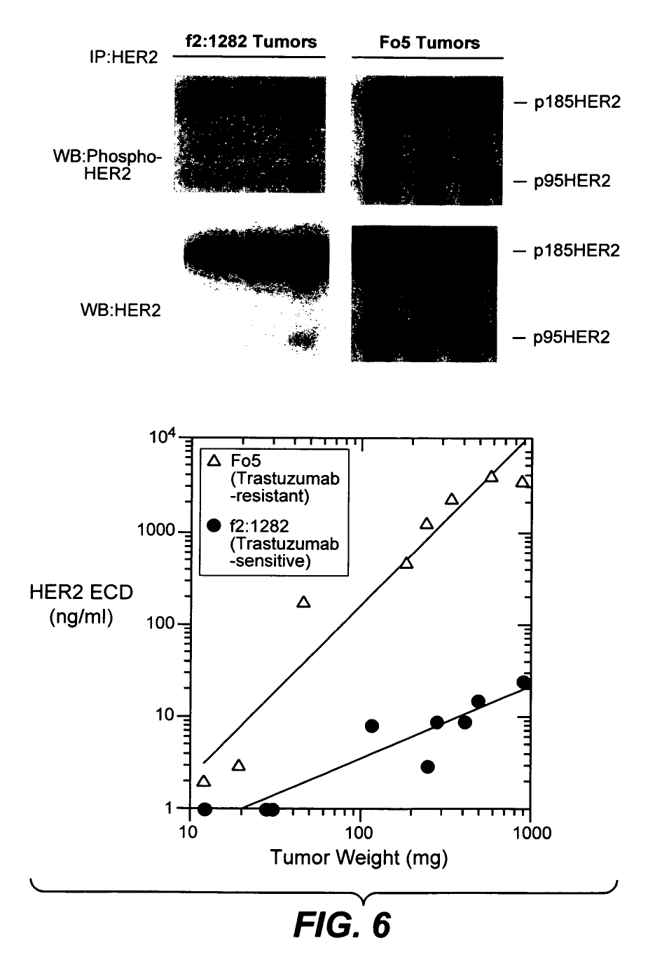

An animal model system where mammary tumors derived from a MMTVHER2 transgenic mouse (Finkle et al. Clin. Cancer Res. 10:2499-2511 (2004)) was used to identify the HER2 sheddase. This animal model system reproducibly shed different levels of HER2 into serum and exhibited differential sensitivity to trastuzumab (FIG. 6, right panel). This difference in the level of serum HER2 also correlated well with the presence of a protein fragment immunoprecipitated from three independent Fo5 tumor cell lysates having an apparent molecular weight of 95 kD, consistent with the size of p95 HER2 (FIG. 6, lower left panel). This fragment is constitutively phosphorylated in the Fo5 tumor cell lysates, indicating that this fragment may have biological activity in these tumors. The increased levels of a HER2 sheddase may contribute to the trastuzumab resistance of this line, since the epitope of trastuzumab would be lost by proteolytic cleavage (Cho et al. Nature 421: 756-760 (2003)).

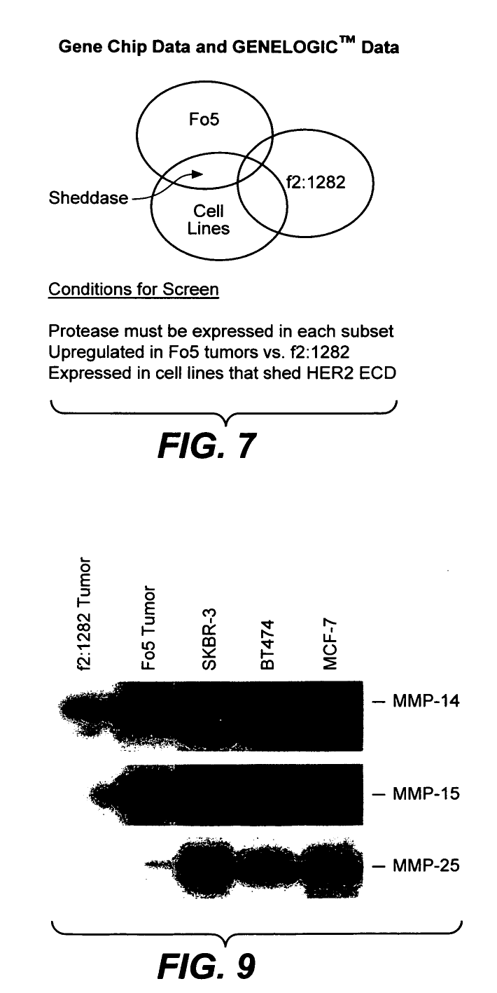

Since the Fo5 tumor line sheds higher levels of HER2 and has high levels of p95HER2 fragment, differential expression analysis was used as an approach to identify transcripts that are upregulated in Fo5 tumors relative to f2:1282 tumors. RNA was prepared from 3 individual tumor samples from either Fo5 tumors or f2:1282 tumors with an average tumor size of 300-600 mm³. RNA from each independent tumor sample was hybridized in triplicate to AFFYMETRIX™ mouse genome single array chip (MOE430P) and known gene array (MOE430A). A Mann-Whitney pairwise comparison was performed identifying 638 upregulated transcripts in Fo5 tumor samples. Since BT 474 and SKBR-3 cell lines also shed HER2 ECD into conditioned medium (FIG. 5, left panel), this list was compared to transcripts that are expressed in both cell lines using the Genelogic Bioexpress database. The approach taken to identify the HER2 sheddase is illustrated in FIG. 7.

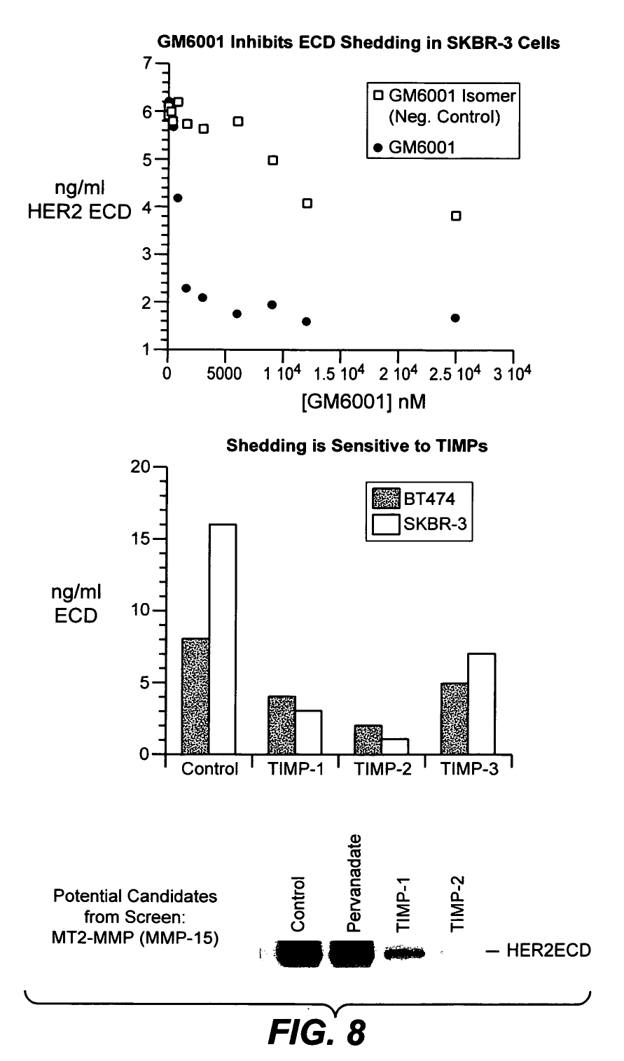

In order to narrow down the number of candidate genes, the general met alloprotease inhibitor GM6001 was used to evaluate whether MMP activity plays a role in regulating HER2 shedding. GM6001, but not its stereoisomer, dose dependently inhibited HER2 shedding in SKBR-3 cells as determined by a HER2 ECD ELISA (FIG. 8, left panel). This result is consistent with previously published reports that the general met alloprotease inhibitor BB-94 inhibited HER2 ECD shedding in breast carcinoma cell lines (Codony-Servat et al. Cancer Res. 59:1196-1201 (1999)). Timp-1, Timp-2 and Timp-3 are naturally occurring peptides that regulate met alloprotease activity (Overall and Lopez-Otin Nature Reviews Cancer 2:657-672 (2002)). Adding purified Timp-1, Timp-2, or Timp-3 to the conditioned media of SKBR-3 cells at a concentration of 1 μg/ml reduced HER2 ECD levels (FIG. 8, right panel). However, Timp-2 reduced HER2 ECD shedding the most efficiently (FIG. 8, right panel). Timp-2 is known to efficiently inhibit the MT-MMP subfamily of met alloproteases (Overall and Lopez-Otin Nature Reviews Cancer 2:657-672 (2002)). These data suggested that the protease responsible for HER2 shedding in SKBR-3 and BT 474 cells is a met alloprotease, significantly reducing the number of candidates identified from the bioinformatics screen.

Several members of the MT-MMP family are expressed in SKBR-3 and BT 474 cells based on the GENELOGIC™ database. Both MTI-MMP and MT2-MMP are also expressed in Fo5 tumors from the AFFYMETRIX™ microarray data. To determine if these transcripts are expressed, Fo5 and f2:1282 tumor and breast carcinoma cell line lysates were immunoblotted with polyclonal antibodies to these MT-MMPs that recognize both human and mouse proteins (FIG. 9). The cell lines all express MMP-14, MMP-15, and MMP-25, at different levels. While MMP-14 is expressed in both f2:1282 and Fo5 tumor cell lysates, MMP-15 is abundantly expressed in Fo5 tumors lysates. This result was consistent with the Mann-Whitney comparison which indicated a 2-fold higher level of mRNA expression of MMP-15 in Fo5 tumors. To verify that MMP-15 was expressed in the epithelial cells of the tumor, and not the surrounding stromal cells, Fo5 and f2:1282 tumors were sectioned and stained with either an anti-HER2 antibody (Dako) or an anti-MMP-15 antibody (Ab-1). Both f2:1282 and Fo5 tumors express human HER2, but MMP-15 is more abundantly expressed in the Fo5 tumors than the f2:1282 tumors.

Substrates for members of the MT-MMPs have traditionally been thought to be extracellular matrix proteins. A biochemical assay was set up to test candidates identified from the microarray data to cleave full length HER2 in vitro. This assay uses a purified recombinant fusion protein as a substrate for candidate proteases and consists of a N-terminally gD epitope-tagged HER2 ECD in-frame with the Fc heavy chain of human IgG. Several different proteins were used in these studies. gDHER2(+)-IgG contains amino acids 2-656 of HER2 ECD (GenBank accession AAA75493) and gDHER2(−)-IgG contains amino acids 2-626 of HER2 ECD. The gDHER2(+)-IgG recombinant protein contains juxtamembrane sequence that have previously been shown to contain a putative HER2 sheddase site (PA/EQR/ASP; SEQ ID NO. 23) while the gDHER2(−)-IgG protein lacks this sequence (Yuan et al. Protein Expression and Purification 29:217-222 (2003)). A truncated version gDHER2(+)-IgG was also used in this assay, gDHER2(DIV)-IgG, which has only domain IV (DIV) of HER2 in-frame with human Fc. Purified catalytic domains of MMP-14, MMP-15, MMP-16, MMP-19, and MMP-25 were incubated with gDHER2(DIV)-IgG for 20 minutes at 37° C. All MT-MMPs except MMP-14 efficiently cleave gDHER2(DIV)-IgG substrate (FIG. 13, left panel). The cleaved protein products were excised and N-terminally sequenced to identify the cleavage site. All four MT-MMPs cleaved HER2 near the published sequence site (Yuan et al. Protein Expression and Purification 29: 217-222 (2003)) (FIG. 13, right panel).

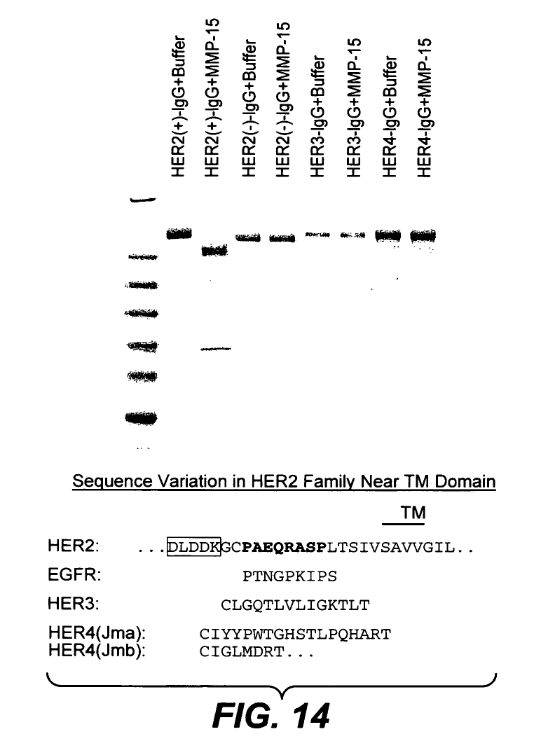

HER2-Fc fusion proteins that have the sequence PINCTHSCVDLDDKGCPAEQRASPASPLTSIV (SEQ ID NO. 21) are substrates for these four MMPs in vitro (FIG. 14). This data suggest that HER2 is a substrate for MMP-15.

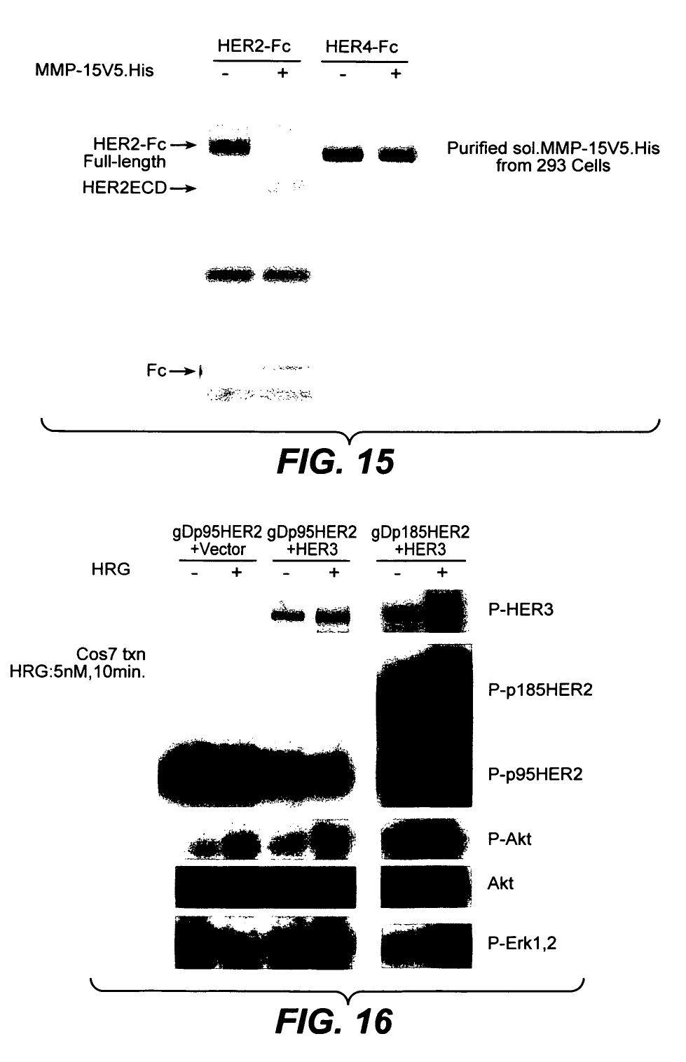

MT-MMP substrate recognition may be influenced by the hemopexin domain (Overall and Lopez-Otin Nature Reviews Cancer 2:657-672 (2002)). A soluble form of MMP-15 with a C-terminal V5 His epitope was transiently expressed in 293 cells and purified from 293 conditioned media by affinity chromatography. As shown in FIG. 15, this soluble form of MMP-15 can also cleave gDHER2(+)-IgG in the in vitro sheddase assay.

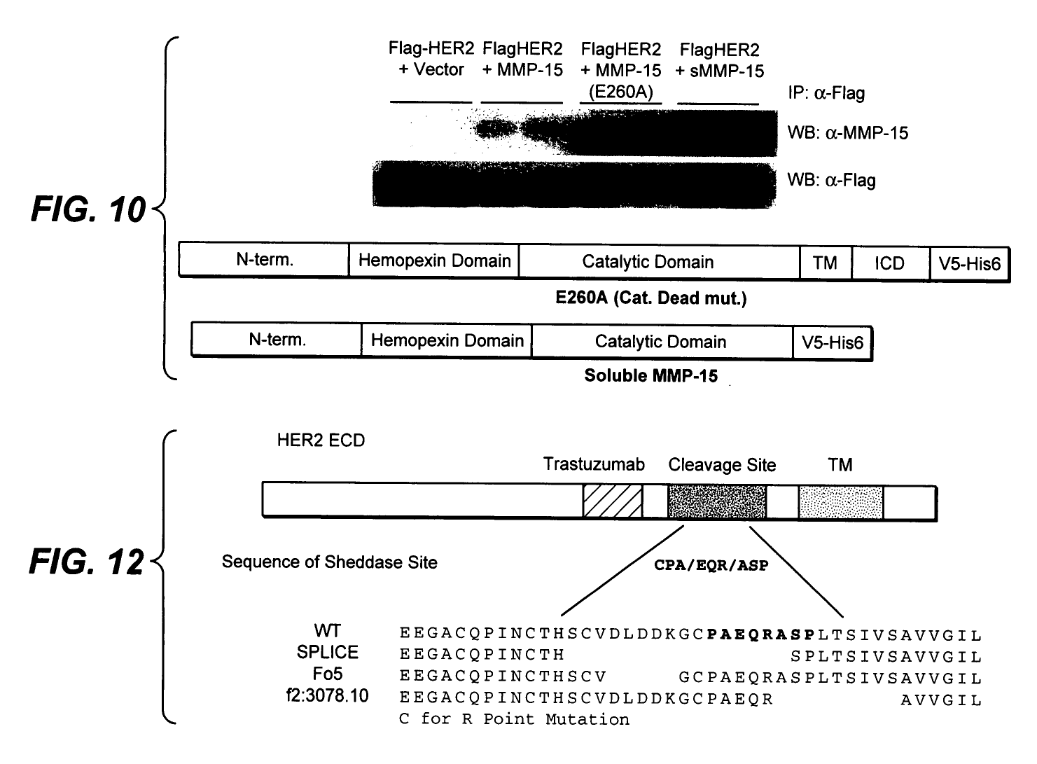

To determine if MMP-15 and HER2 can associate in the membrane, co-immunoprecipitation assays were performed using transiently transfected Cos-7 cells. pRK5.FlagHER2 was co-transfected with either vector, pcDNA3.MMP-15V5His, pcDNA3.sMMP-15V5His, or pcDNA3.MMP-15(E260A). FlagHER2 was immunoprecipitated with an anti-FLAG resin. FIG. 10 shows that both soluble MMP-15 and a catalytically dead (E260A) mutant can associate with Flag-HER2. This is consistent with the notion that MMP-15 can cleave HER2 in membranes since the wild-type version of the protease, pcDNA3.MMP-15V5His, would remove the FlagHER2 ECD and a HER2-MMP-15 complex would not be co-immunoprecipitated.

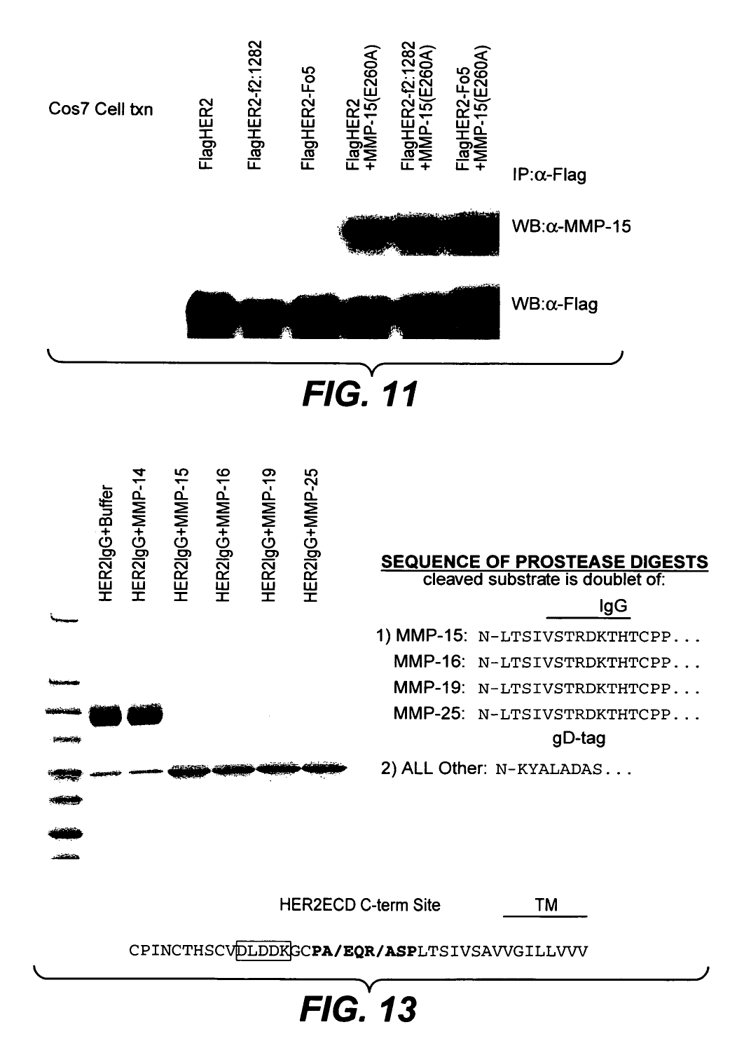

Somatic point mutations and deletions and splice variants have been identified mammary tumors from MMTVHER2 transgenic animals (Siegel et al. EMBO J. 18:2149-2164 (1999)); and Finkle et al. Clin. Cancer Res. 10:2499-2511 (2004)). These mutations most frequently occur in the HER2 extracellular domain near the transmembrane domain. The Fo5 tumor line has a five amino acid deletion DLDDK (SEQ ID NO. 22) that is adjacent to the HER2 cleavage site while the f2:1282 tumor line has a single point mutation C for R (FIG. 12). These mutations do not influence MMP-15 association (FIG. 11). However, it is possible that the proximity of the DLDDK (SEQ ID NO. 22) deletion to the HER2 MMP cleavage site may influence enzymatic activity of the protease.

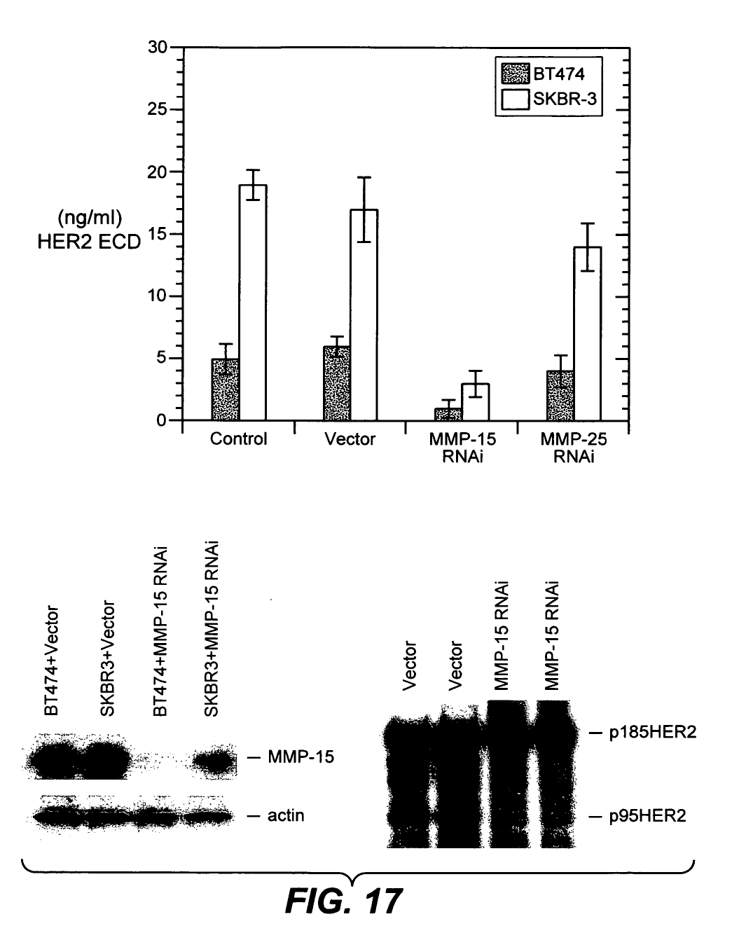

RNA inhibition (RNAi) is a method that can downregulate target transcript and protein levels. To examine the biological role of MMP-15 in SKBR-3 and BT 474 cells, an anti-MMP-15 RNAi construct was used. When introduced into SKBR-3 or BT 474 cells, this shRNAi expression plasmid reduced endogenous protein levels of MMP-15 in both cell types (FIG. 17, left upper panel). Transfection of this shRNA also reduced the amount of p95 HER2 in both cell lines (FIG. 17, left lower panel). Loss of MMP-15 protein in these cells significantly reduced HER2 ECD shedding in both lines (FIG. 17, right panel). However, no significant reduction in HER2 ECD shedding was observed when a shRNAi to MMP-25 was introduced into either cell line (FIG. 17, right panel). These experiments strongly suggest that loss of MMP-15 dramatically effects HER2 ECD shedding.

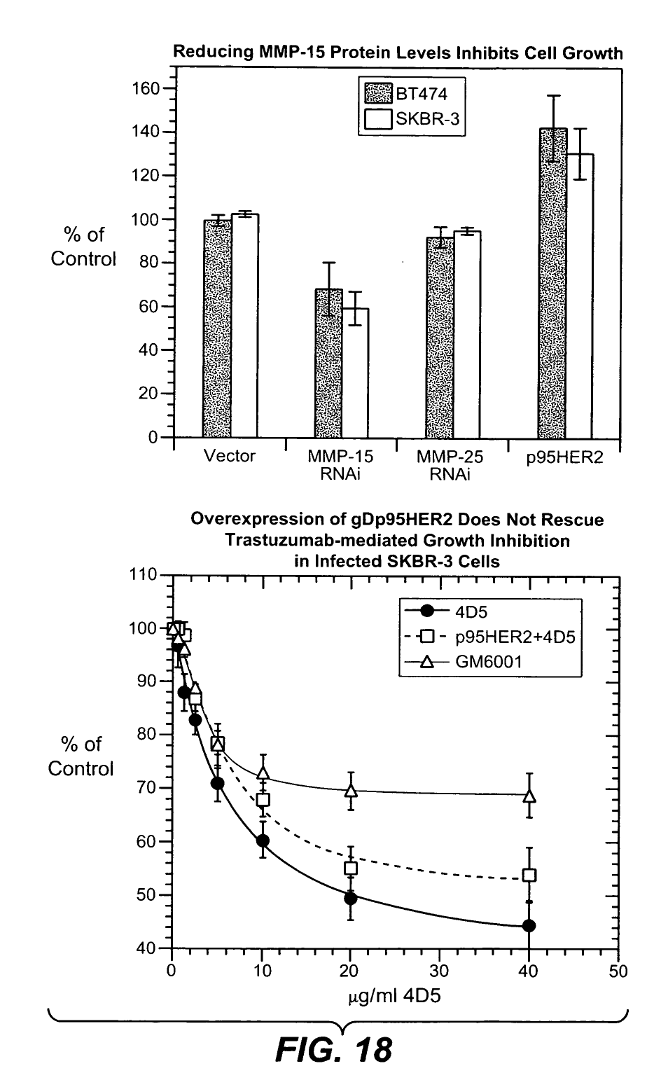

Reducing MMP-15 protein levels by RNAi also has an effect on the growth rates of SKBR-3 and BT 474 cells (FIG. 18, left panel). This effect is also duplicated by a shRNAi to MMP-25. This suggests that reducing HER2 ECD shedding also downregulates the amount of p95 HER2 in BT 474 and SKBR-3 cells, thereby reducing cell growth rates. When a gD-tagged p95 HER2 construct was introduced into these cell lines, an increase in cell growth rates was detected in both cell types relative to control transfected cells (FIG. 18, left panel). This construct is constituively phosphorylated and activates MAPK when introduced into Cos-7 cells in a ligand-independent manner (FIG. 16). However, this construct must still heterodimerize with HER3 or EGFR in order to activate Akt signaling pathways consistent with a previously published study (Xia et al. Oncogene 23:646-653 (2004)). Overexpression of gDp95HER2 in SKBR-3 cells did not significantly inhibit trastuzumab-mediated growth inhibition in these cells (FIG. 18, right panel).

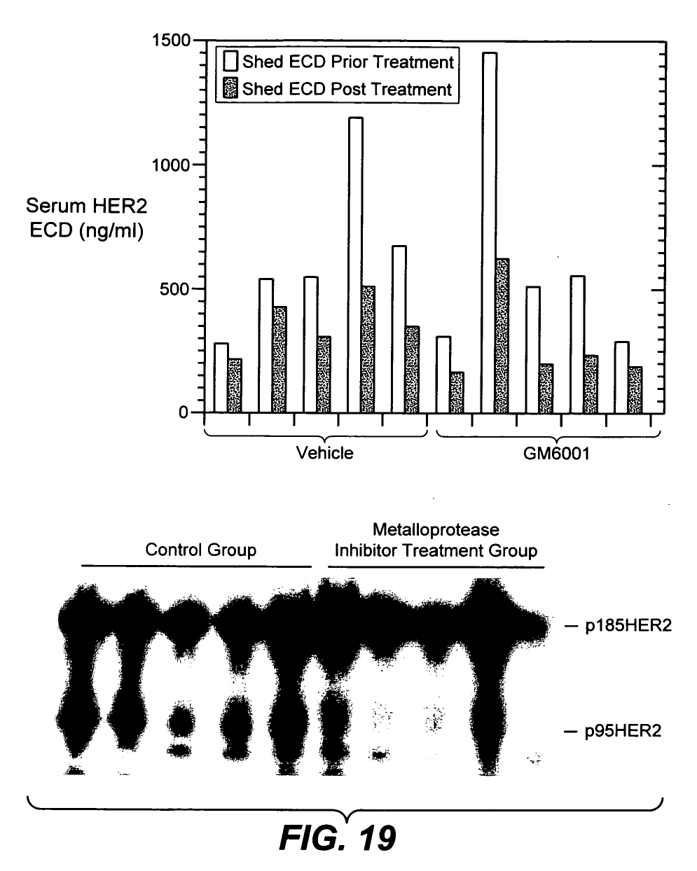

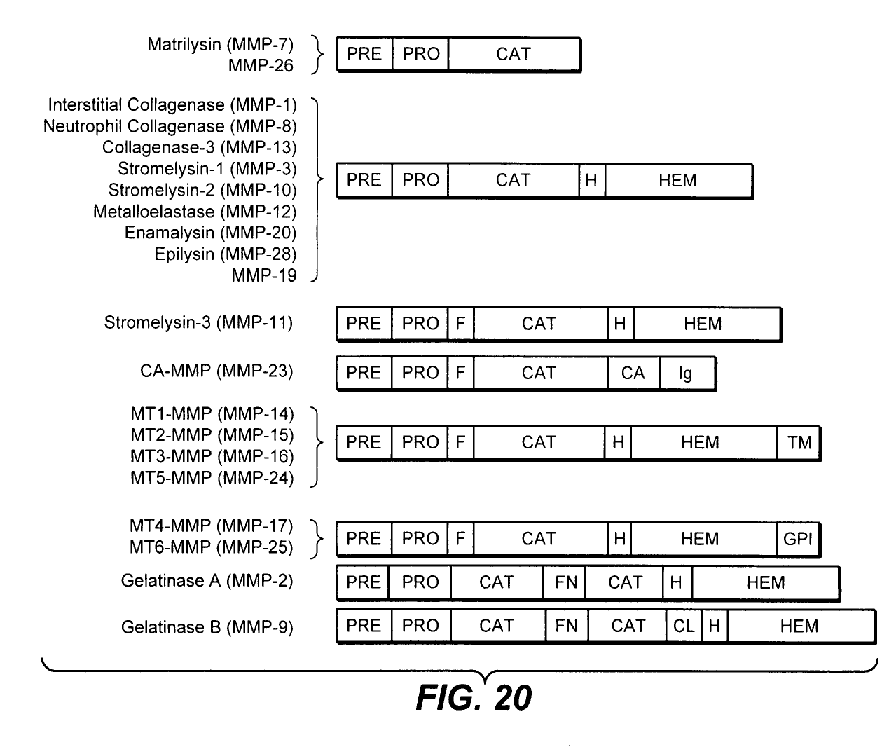

A pharmacological approach was used to determine if MMP activity plays a role in regulating HER2 shedding and p95 HER2 levels in Fo5 tumors. FVB mice with FoS tumors with average size of 400-600 mm³ were injected with GM6001 intraperitoneally or control vehicle daily over the course of three days. On day four, serum was collected and assayed for HER2 ECD by ELISA, and tumors collected and weighed. HER2 was immunoprecipitated from tumor cell lysates and assayed for p95 HER2 levels by Western blotting. GM6001 treated animals showed a significant reduction in the amount of p95 HER2 as compared to control animals (FIG. 19, right panel). Serum HER2 ECD levels were reduced in both vehicle and GM6001 treated animals (FIG. 19, left panel), however, a greater reduction was observed in GM6001 treated animals.

### SUMMARY

The experiments above demonstrate that the matrix met alloprotease MMP-15 cleaves HER2 in vitro at a site that is consistent with purified shed ECD from SKBR3 cells, and interacts with full length HER2. Reducing levels of this MMP correlates well with reduced HER2 receptor shedding, and decreases basal proliferation levels in SKBR3 and BT474 cell lines. Trastuzumab appears to inhibit HER2 receptor shedding by indirectly regulating MMP-15 activity, but does not compete with MMP-15 for substrate binding and cleavage. Reducing HER2 shedding with a MMP antagonist, such as an MMP-15 antagonist, represents a therapeutic approach for treating cancer, including trastuzumab-resistant cancer.

What is claimed is:

1. A method for inhibiting HER2 shedding comprising treating a HER2 expressing cell with a matrix met alloprotease (MMP) antagonist in an amount effective to inhibit HER2 shedding.

2. The method of claim 1 wherein the MMP antagonist is a membrane-tethered MMP (MT-MMP) antagonist.

3. The method of claim 2 wherein the MT-MMP is selected from the group consisting of MMP-15 (MT2-MMP), MMP-16 (MT3-MMP), MMP-24 (MT5-MMP), MMP-17 (MT4-MMP), and MMP-25 (MT6-MMP).

4. The method of claim 3 wherein the MT-MMP is MMP-15.

5. The method of claim 1 wherein the cell displays HER2 overexpression, amplification, or activation.

6. The method of claim 5 wherein the cell displays HER2 overexpression or amplification.

7. The method of claim 1 further comprising treating the cell with a HER inhibitor.

8. The method of claim 7 wherein the HER inhibitor is a HER2 antibody.

9. The method of claim 8 wherein the HER2 antibody is trastuzumab or pertuzumab.

10. The method of claim 7 wherein the HER inhibitor is selected from the group consisting of trastuzumab, pertuzumab, cetuximab, ABX-EGF, EMD7200, gefitinib, erlotinib, CP724714, CI1033, GW572016, IMC-11F8, and TAK165.

11. A method for reducing HER2 extracellular domain (ECD) serum level in a mammal, comprising administering a matrix met alloprotease (MMP) antagonist to the mammal in an amount effective to reduce the HER2 ECD serum level in the mammal.

12. The method of claim 11 wherein the mammal has an elevated MMP level.

13. A method for treating cancer in a mammal comprising administering a matrix met alloprotease (MMP) antagonist to the mammal in an amount effective to treat the cancer.

14. The method of claim 13 wherein the cancer displays HER expression, amplification, or activation.

15. The method of claim 14 wherein the cancer displays HER2 overexpression or amplification.

16. The method of claim 13 wherein the mammal has an elevated shed HER2 serum level or elevated p95 HER2 level.

17. A method for treating a HER inhibitor-resistant cancer in a mammal comprising administering to the mammal a matrix met alloprotease (MMP) antagonist in an amount effective to treat the cancer.

18. The method of claim 17 wherein the HER inhibitor is trastuzumab.

19. A method for reducing p95 HER2 level in a cell comprising exposing the cell to a matrix met alloprotease (MMP) antagonist in an amount effective to reduce the p95 HER2 level.

20. A method of diagnosis comprising evaluating MMP-15 (MT2-MMP) in a sample from a cancer patient, wherein elevated MMP-15 level or activity indicates the patient has an elevated p95 HER2 or shed HER2 serum level, or will have a poor clinical outcome.

21. The method of claim 20 wherein an elevated MMP-15 level indicates the patient will have a poor clinical outcome.
